SEQUENCE LISTING (22 sequences)

SEQ ID NO: 1 - DNA, 1277 bp, Gallus gallus
atggtcgaaatgctgctgttgacaagaattctcttggtgggcttcatctgcgctcttttagtctcctctgggctgacttgtggaccaggcaggggcattggaaaaaggaggcaccccaaaaagctgaccccgttagcctataagcagtttattcccaatgtggcagagaagaccctaggggccagtggaagatatgaagggaagatcacaagaaactccgagagatttaaagaactaaccccaaattacaaccctgacattatttttaaggatgaagagaacacgggagctgacagactgatgactcagcgctgcaaggacaagctgaatgccctggcgatctcggtgatgaaccagtggcccggggtgaagctgcgggtgaccgagggctgggacgaggatggccatcactccgaggaatcgctgcactacgagggtcgcgccgtggacatcaccacgtcggatcgggaccgcagcaagtacggaatgctggcccgcctcgccgtcgaggccggcttcgactgggtctactacgagtccaaggcgcacatccactgctccgtcaaagcagaaaactcagtggcagcgaaatcaggaggctgcttccctggctcagccacagtgcacctggagcatggaggcaccaagctggtgaaggacctgagccctggggaccgcgtgctggctgctgacgcggacggccggctgctctacagtgacttcctcaccttcctcgaccggatggacagctcccgaaagctcttctacgtcatcgagacgcggcagccccgggcccggctgctactgacggcggcccacctgctctttgtggccccccagcacaaccagtcggaggccacagggtccaccagtggccaggcgctcttcgccagcaacgtgaagcctggccaacgtgtctatgtgctgggcgagggcgggcagcagctgctgccggcgtctgtccacagcgtctcattgcgggaggaggcgtccggagcctacgccccactcaccgcccagggcaccatcctcatcaaccgggtgttggcctcctgctacgccgtcatcgaggagcacagttgggcccattgggccttcgcaccattccgcttggctcaggggctgctggccgccctctgcccagatggggccatccctactgccgccaccaccaccactggcatccattggtactcacggctcctctaccgcatcggcagctgggtgctggatggtgacgcgctgcatccgctgggcatggtggcaccggccagctg

SEQ ID NO: 2 - DNA, 1190 bp, Mus musculus
atggctctgccggccagtctgttgcccctgtgctgcttggcactcttggcactatctgcccagagctgcgggccgggccgaggaccggttggccggcggcgttatgtgcgcaagcaacttgtgcctctgctatacaagcagtttgtgcccagtatgcccgagcggaccctgggcgcgagtgggccagcggaggggagggtaacaagggggtcggagcgcttccgggacctcgtacccaactacaaccccgacataatcttcaaggatgaggagaacagcggcgcagaccgcctgatgacagagcgttgcaaagagcgggtgaacgctctagccatcgcggtgatgaacatgtggcccggagtacgcctacgtgtgactgaaggctgggacgaggacggccaccacgcacaggattcactccactacgaaggccgtgccttggacatcaccacgtctgaccgtgaccgtaataagtatggtttgttggcgcgcctagctgtggaagccggattcgactgggtctactacgagtcccgcaaccacatccacgtatcggtcaaagctgataactcactggcggtccgagccggaggctgctttccgggaaatgccacggtgcgcttgcggagcggcgaacggaaggggctgagggaactacatcgtggtgactgggtactggccgctgatgcagcgggccgagtggtacccacgccagtgctgctcttcctggaccgggatctgcagcgccgcgcctcgttcgtggctgtggagaccgagcggcctccgcgcaaactgttgctcacaccctggcatctggtgttcgctgctcgcgggccagcgcctgctccaggtgactttgcaccggtgttcgcgcgccgcttacgtgctggcgactcggtgctggctcccggcggggacgcgctccagccggcgcgcgtagcccgcgtggcgcgcgaggaagccgtgggcgtgttcgcaccgctcactgcgcacgggacgctgctggtcaacgacgtcctcgcctcctgctacgcggttctagagagtcaccagtgggcccaccgcgccttcgcccctttgcggctgctgcacgcgctcggggctctgctccctgggggtgcagtccagccgactggcatgcattggtactctcgcctcctttaccgcttggccgaggagttaatgggctg

SEQ ID NO: 3 - DNA, 1281 bp, Mus musculus
atgtctcccgcctggctccggccccgactgcggttctgtctgttcctgctgctgctgcttctggtgccggcggcgcggggctgcgggccgggccgggtggtgggcagccgccggaggccgcctcgcaagctcgtgcctcttgcctacaagcagttcagccccaacgtgccggagaagaccctgggcgccagcgggcgctacgaaggcaagatcgcgcgcagctctgagcgcttcaaagagctcacccccaactacaatcccgacatcatcttcaaggacgaggagaacacgggtgccgaccgcctcatgacccagcgctgcaaggaccgtctgaactcactggccatctctgtcatgaaccagtggcctggtgtgaaactgcgggtgaccgaaggccgggatgaagatggccatcactcagaggagtctttacactatgagggccgcgcggtggatatcaccacctcagaccgtgaccgaaataagtatggactgctggcgcgcttagcagtggaggccggcttcgactgggtgtattacgagtccaaggcccacgtgcattgctctgtcaagtctgagcattcggccgctgccaagacaggtggctgctttcctgccggagcccaggtgcgcctagagaacggggagcgtgtggccctgtcagctgtaaagccaggagaccgggtgctggccatgggggaggatgggacccccaccttcagtgatgtgcttattttcctggaccgcgagccaaaccggctgagagctttccaggtcatcgagactcaggatcctccgcgtcggctggcgctcacgcctgcccacctgctcttcattgcggacaatcatacagaaccagcagcccacttccgggccacatttgccagccatgtgcaaccaggccaatatgtgctggtatcaggggtaccaggcctccagcctgctcgggtggcagctgtctccacccacgtggcccttgggtcctatgctcctctcacaaggcatgggacacttgtggtggaggatgtggtggcctcctgctttgcagctgtggctgaccaccatctggctcagttggccttctggcccctgcgactgtttcccagtttggcatggggcagctggaccccaagtgagggtgttcactcctaccctcagatgctctaccgcctggggcgtctcttgctagaagagagcaccttccatccactgggcatgtctggggcaggaagctgaagggactctaaccactgccctcctggaactgctgtgcgtggatcc

SEQ ID NO: 4 - DNA, 1313 bp, Mus musculus
atgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaggctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctg

SEQ ID NO: 5 - DNA, 1256 bp, Brachydanio rerio
atgcggcttttgacgagagtgctgctggtgtctcttctcactctgtccttggtggtgtccggactggcctgcggtcctggcagaggctacggcagaagaagacatccgaagaagctgacacctctcgcctacaagcagttcatacctaatgtcgcggagaagaccttaggggccagcggcagatacgagggcaagataacgcgcaattcggagagatttaaagaacttactccaaattacaatcccgacattatctttaaggatgaggagaacacgggagcggacaggctcatgacacagagatgcaaagacaagctgaactcgctggccatctctgtaatgaaccactggccaggggttaagctgcgtgtgacagagggctgggatgaggacggtcaccattttgaagaatcactccactacgagggaagagctgttgatattaccacctctgaccgagacaagagcaaatacgggacactgtctcgcctagctgtggaggctggatttgactgggtctattacgagtccaaagcccacattcattgctctgtcaaagcagaaaattcggttgctgcgaaatctgggggctgtttcccaggttcggctctggtctcgctccaggacggaggacagaaggccgtgaaggacctgaaccccggagacaaggtgctggcggcagacagcgcgggaaacctggtgttcagcgacttcatcatgttcacagaccgagactccacgacgcgacgtgtgttttacgtcatagaaacgcaagaacccgttgaaaagatcaccctcaccgccgctcacctcctttttgtcctcgacaactcaacggaagatctccacaccatgaccgccgcgtatgccagcagtgtcagagccggacaaaaggtgatggttgttgatgatagcggtcagcttaaatctgtcatcgtgcagcggatatacacggaggagcagcggggctcgttcgcaccagtgactgcacatgggaccattgtggtcgacagaatactggcgtcctgttacgccgtaatagaggaccaggggcttgcgcatttggccttcgcgcccgccaggctctattattacgtgtcatcattcctgtcccccaaaactccagcagtcggtccaatgcgactttacaacaggagggggtccactggtactccaggctcctgtcatcaaatgggaacgtggcttttggacagcaacatgcttcatcctttggggatgtcagtaaactcaagctg

SEQ ID NO: 6 - DNA, 1425 bp, Homo sapiens
  modified_base (1387...1389): n=a, c, g, or t
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagcnnnagccggggggccgggggaggggcgcgggagggggcc

SEQ ID NO: 7 - DNA, 1622 bp, Homo sapiens
catcagcccaccaggagacctcgcccgccgctcccccgggctccccggccatgtctcccgcccggctccggccccgactgcacttctgcctggtcctgttgctgctgctggtggtgcccgcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagccccacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccgcgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattccc

SEQ ID NO: 8 - DNA, 1191 bp, Homo sapiens
atggctctcctgaccaatctactgcccttgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgcaaggagagggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctga

SEQ ID NO: 9 - DNA, 1251 bp, Brachydanio rerio
atggacgtaaggctgcatctgaagcaatttgctttactgtgttttatcagcttgcttctgacgccttgtggattagcctgtggtcctggtagaggttatggaaaacgaagacacccaaagaaattaaccccgttggcttacaagcaattcatccccaacgttgctgagaaaacgcttggagccagcggcaaatacgaaggcaaaatcacaaggaattcagagagatttaaagagctgattccgaattataatcccgatatcatctttaaggacgaggaaaacacaaacgctgacaggctgatgaccaagcgctgtaaggacaagttaaattcgttggccatatccgtcatgaaccactggcccggcgtgaaactgcgcgtcactgaaggctgggatgaggatggtcaccatttagaagaatctttgcactatgagggacgggcagtggacatcactacctcagacagggataaaagcaagtatgggatgctatccaggcttgcagtggaggcaggattcgactgggtctattatgaatctaaagcccacatacactgctctgtcaaagcagaaaattcagtggctgctaaatcaggaggatgttttcctgggtctgggacggtgacacttggtgatgggacgaggaaacccatcaaagatcttaaagtgggcgaccgggttttggctgcagacgagaagggaaatgtcttaataagcgactttattatgtttatagaccacgatccgacaacgagaaggcaattcatcgtcatcgagacgtcagaacctttcaccaagctcaccctcactgccgcgcacctagttttcgttggaaactcttcagcagcttcgggtataacagcaacatttgccagcaacgtgaagcctggagatacagttttagtgtgggaagacacatgcgagagcctcaagagcgttacagtgaaaaggatttacactgaggagcacgagggctcttttgcgccagtcaccgcgcacggaaccataatagtggatcaggtgttggcatcgtgctacgcggtcattgagaaccacaaatgggcacattgggcttttgcgccggtcaggttgtgtcacaagctgatgacgtggctttttccggctcgtgaatcaaacgtcaattttcaggaggatggtatccactggtactcaaatatgctgtttcacatcggctcttggctgctggacagagactctttccatccactcgggattttacacttaagttga

SEQ ID NO: 10 - protein, 425 aa, Gallus gallus
MVEMLLLTRILLVGFICALLVSSGLTCGPGRGIGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEHGGTKLVKDLSPGDRVLAADADGRLLYSDFLTFLDRMDSSRKLFYVIETRQPRARLLLTAAHLLFVAPQHNQSEATGSTSGQALFASNVKPGQRVYVLGEGGQQLLPASVHSVSLREEASGAYAPLTAQGTILINRVLASCYAVIEEHSWAHWAFAPFRLAQGLLAALCPDGAIPTAATTTTGIHWYSRLLYRIGSWVLDGDALHPLGMVAPAS

SEQ ID NO: 11 - protein, 396 aa, Mus musculus [sequence omitted]

SEQ ID NO: 12 - protein, 411 aa, Mus musculus
MSPAWLRPRLRFCLFLLLLLLVPAARGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGRDEDGHHSEESLHYEGRAVDITTSDRDRNKYGLLARLAVEAGFDWVYYESKAHVHCSVKSEHSAAAKTGGCFPAGAQVRLENGERVALSAVKPGDRVLAMGEDGTPTFSDVLIFLDREPNRLRAFQVIETQDPPRRLALTPAHLLFIADNHTEPAAHFRATFASHVQPGQYVLVSGVPGLQPARVAAVSTHVALGSYAPLTRHGTLVVEDVVASCFAAVADHHLAQLAFWPLRLFPSLAWGSWTPSEGVHSYPQMLYRLGRLLLEESTFHPLGMSGAGS

SEQ ID NO: 13 - protein, 437 aa, Mus musculus
MLLLLARCFLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVRLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 14 - protein, 418 aa, Brachydanio rerio
MRLLTRVLLVSLLTLSLVVSGLACGPGRGYGRRRHPKKLTPLAYKQFIPNVAEKTLGASGRYEGKITRNSERFKELTPNYNPDIIFKDEENTGADRLMTQRCKDKLNSLAISVMNHWPGVKLRVTEGWDEDGHHFEESLHYEGRAVDITTSDRDKSKYGTLSRLAVEAGFDWVYYESKAHIHCSVKAENSVAAKSGGCFPGSALVSLQDGGQKAVKDLNPGDKVLAADSAGNLVFSDFIMFTDRDSTTRRVFYVIETQEPVEKITLTAAHLLFVLDNSTEDLHTMTAAYASSVRAGQKVMVVDDSGQLKSVIVQRIYTEEQRGSFAPVTAHGTIVVDRILASCYAVIEDQGLAHLAFAPARLYYYVSSFLSPKTPAVGPMRLYNRRGSTGTPGSCHQMGTWLLDSNMLHPLGMSVNSS

SEQ ID NO: 15 - protein, 475 aa, Homo sapiens
  SITE (463): Xaa=unknown amino acid residue
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSSXSRGAGGGAREGA

SEQ ID NO: 16 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 17 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 18 - protein, 416 aa, Brachydanio rerio
MDVRLHLKQFALLCFISLLLTPCGLACGPGRGYGKRRHPKKLTPLAYKQFIPNVAEKTLGASGKYEGKITRNSERFKELIPNYNPDIIFKDEENTNADRLMTKRCKDKLNSLAISVMNHWPGVKLRVTEGWDEDGHHLEESLHYEGRAVDITTSDRDKSKYGMLSRLAVEAGFDWVYYESKAHIHCSVKAENSVAAKSGGCFPGSGTVTLGDGTRKPIKDLKVGDRVLAADEKGNVLISDFIMFIDHDPTTRRQFIVIETSEPFTKLTLTAAHLVFVGNSSAASGITATFASNVKPGDTVLVWEDTCESLKSVTVKRIYTEEHEGSFAPVTAHGTIIVDQVLASCYAVIENHKWAHWAFAPVRLCHKLMTWLFPARESNVNFQEDGIHWYSNMLFHIGSWLLDRDSFHPLGILHLS

SEQ ID NO: 19 - DNA, 1416 bp, Drosophila melanogaster
  CDS (1)..(1413)
atggataaccacagctcagtgccttgggccagtgccgccagtgtcacctgtctctccctgggatgccaaatgccacagttccagttccagttccagctccaaatccgcagcgagctccatctccgcaagcccgcaagaagaacgcaaacgatgcgccacattgcgcatacgcagcgttgcctcagcaggctgacctctctggtggccctgctgctgatcgtcttgccgatggtctttagcccggctcacagctgcggtcctggccgaggattgggtcgtcatagggcgcgcaacctgtatccgctggtcctcaagcagacaattcccaatctatccgagtacacgaacagcgcctccggacctctggagggtgtgatccgtcgggattcgcccaaattcaaggacctcgtgcccaactacaacagggacatccttttccgtgacgaggaaggcaccggagcggatggcttgatgagcaagcgctgcaaggagaagctaaacgtgctggcctactcggtgatgaacgaatggcccggcatccggctgctggtcaccgagagctgggacgaggactaccatcacggccaggagtcgctccactacgagggccgagcggtgaccattgccacctccgatcgcgaccagtccaaatacggcatgctcgctcgcctggccgtcgaggctggattcgattgggtctcctacgtcagcaggcgccacatctactgctccgtcaagtcagattcgtcgatcagttcccacgtgcacggctgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcgcatcgcatcgaggagaagaaccaggtgctcgtacgggatgtggagacgggcgagctgaggccccagcgagtggtcaagttgggcagtgtgcgcagtaagggcgtggtcgcgccgctgacccgcgagggcaccattgtggtcaactcggtggccgccagttgctatgcggtgatcaacagtcagtcgctggcccactggggactggctcccatgcgcctgctgtccacgctggaggcgtggctgcccgccaaggagcagttgcacagttcgccgaaggtggtgagctcggcgcagcagcagaatggcatccattggtatgccaatgcgctctacaaggtcaaggactacgtgctgccgcagagctggcgccacgattga

SEQ ID NO: 20 - protein, 471 aa, Drosophila melanogaster
MDNHSSVPWASAASVTCLSLGCQMPQFQFQFQLQIRSELHLRKPARRTQTMRHIAHTQRCLSRLTSLVALLLIVLPMVFSPAHSCGPGRGLGRHRARNLYPLVLKQTIPNLSEYTNSASGPLEGVIRRDSPKFKDLVPNYNRDILFRDEEGTGADGLMSKRCKEKLNVLAYSVMNEWPGIRLLVTESWDEDYHHGQESLHYEGRAVTIATSDRDQSKYGMLARLAVEAGFDWVSYVSRRHIYCSVKSDSSISSHVHGCFTPESTALLESGVRKPLGELSIGDRVLSMTANGQAVYSEVILFMDRNLEQMQNFVQLHTDGGAVLTVTPAHLVSVWQPESQKLTFVFAHRIEEKNQVLVRDVETGELRPQRVVKLGSVRSKGVVAPLTREGTIVVNSVAASCYAVINSQSLAHWGLAPMRLLSTLEAWLPAKEQLHSSPKVVSSAQQQNGIHWYANALYKVKDYVLPQSWRHD

SEQ ID NO: 21 - protein, 221 aa, Artificial Sequence
  Description of Artificial Sequence Degenerate Shh polypeptide general formula
CGPGRGXGXRRHPKKLTPLAYKQFIPNVAEKTLGASGRYEGKIXRNSERFKELTPNYNPDIIFKDEENTGADRLMTQRCKDKLNXLAISVMNXWPGVXLRVTEGWDEDGHHXEESLHYEGRAVDITTSDRDXSKYGXLXRLAVEAGFDWVYYESKAHIHCSVKAENSVAAKSGGCFPGSAXVXLXXGGXKXVKDLXPGDXVLAADXXGXLXXSDFXXFXDR

SEQ ID NO: 22 - protein, 167 aa, Artificial Sequence
  Description of Artificial Sequence Degenerate hedgehog polypeptide general formula
CGPGRGXXXRRXXXPKXLXPLXYKQFXPXXXEXTLGASGXXEGXXXRXSERFXXLTPNYNPDIIFKDEENXGADRLMTXRCKXXXNXLAISVMNXWPGVXLRVTEGXDEDGHHXXXSLHYEGRAXDITTSDRDXXKYGXLXRLAVEAGFDWVYYESXXHXHXSVKXX

(19) United States
(12) Patent Application Publication
(10) Pub. No.: US 2003/0104995 A1
(43) Pub. Date: Jun. 5, 2003

(54) Neuroprotective methods and compositions

(76) Inventor: Jennifer Ott Reilly, Andover, MA (US)

Correspondence Address: ROPES & GRAY, ONE INTERNATIONAL PLACE, BOSTON, MA, 02110-2624, US

(21) Appl. No.: 09/845,025

(22) Filed: Apr. 27, 2001

Related U.S. Application Data
(60) Provisional application No. 60/200,765, filed on Apr. 28, 2000.

(51) Int. Cl. A61K 48/00; A61K 38/17

(52) U.S. Cl. 514/12; 514/44; 424/93.2

(57) ABSTRACT

The invention concerns a method of ameliorating a neurodegenerative condition by administration of a combination of a hedgehog therapeutic and a neurotrophic factor. The hedge hog therapeutic can be any ligand that is an agonist for the patched-smothened receptor complex or any compound that causes the production of such a ligand. The neurotrophic factor can be selected from IGF-1, NGF, BDNF, CNTF and others, with NGF being preferred. The neurodegenerative conditions susceptible to amelioration include Alzheimer's Disease, Parkinson's Disease and Huntingdon's Chorea. In an alternative embodiment the invention concerns a kit comprising the hedgehog therapeutic, the neurotrophic factor and a label indicating the above-noted therapeutic use of the kit.

### BACKGROUND OF THE INVENTION

The formation of contacts between developing axons and their targets is a key element in the establishment of proper neuronal architecture. The atrophy of unused synapses is equally important in the development and maintenance of the nervous system. On a larger scale, this process results in the culling of incorrectly connected neurons and the selection of properly connected neurons. Neuronal cell atrophy and death occurs in a variety of diseases, including Alzheimer's disease, Parkinson's disease, Huntington's disease, amyotrphic lateral sclerosis, and peripheral neuropathy.

Alzheimer's Disease is marked by widespread neurodegeneration in the brain including an enhanced loss of the cholinergic neurons that reside in the basal forebrain. The loss of the basal forebrain cholinergic neurons contributes to the cognitive and spatial memory deficits in Alzheimer's diseased patients (Gilmor et al., 1999; Lehericy et al. 1993). Restoring and modulating cholinergic function in Alzheimer's patients is a candidate treatment for the disease (Sramek and Cutler, 1999; Mufson et al., 1998). Other neural cell types may also be involved with the disease.

Parkinson's disease is fairly common, estimates of its incidence varying from 0.1 to 1.0% of the population. It is also of considerable interest for a number of other reasons. First, the disease seems related to the degeneration of the substantia nigra, and to the loss of the neurotransmitter substance dopamine, which is produced by cells of this nucleus. The disease, therefore, provides an important insight into the role of this brainstem nucleus and its neurotransmitter in the control of movement.

The excessive or inappropriate stimulation of excitatory amino acid receptors can lead to neuronal cell damage or loss by way of a mechanism known as excitotoxicity. For instance, excitotoxic action may be responsible for neuronal loss in stroke, cerebral palsy, epilepsy, ageing and Alzheimer's disease, Huntington's disease, and other chronic degenerative disorders. The medical consequences of such neuronal degeneration makes the abatement of these degenerative neurological processes an important therapeutic goal.

The development of neuroprotective agents for the prevention of neuronal loss in acute conditions such as stroke and epilepsy or chronic neurodegenerative disorders including Parkinson's disease, Alzheimer's disease, Huntington's chorea, and motor neuron disease has in fact focused on drugs that inhibit excitatory amino acid neurotransmission or exhibit antioxidant properties. Unfortunately, potent antagonists of the N-methyl-D-aspartate (NMDA) type glutamate receptor, which is thought to mediate excitotoxic neuronal injury, e.g., MK-801 or phencyclidine (PCP), share a high probability of inducing psychotomimetic side effects. Further, these drugs have been associated with acute neurotoxicity in vitro and in vivo, precluding their clinical use.

There are, however, several disadvantages associated with the use of nerve growth factors for treating neurological diseases. They do not readily cross the blood-brain barrier. They are unstable in plasma. And they have poor drug delivery properties. Recently, small molecules have been shown to stimulate neurite outgrowth in vivo. In individuals suffering from a neurological disease, this stimulation of neurite outgrowth protects neurons from further degeneration, and accelerates the regeneration of nerve cells. For example, estrogen has been shown to promote the growth of axons and dendrites, which are neurites sent out by nerve cells to communicate with each other in a developing or injured adult brain >(C. Dominique Toran-Allerand et al., J. Steroid Biochem. Mol. Biol., 56, pp. 169-78 (1996); and B. S. McEwen et al., Brain Res. Dev. Brain. Res., 87, pp. 91-95 (1995). The progress of Alzheimer's disease is slowed in women who take estrogen. Estrogen is hypothesized to complement NGF and other neurotrophins and thereby help neurons differentiate and survive.

Tacrolimus, an immunosuppressive drug, has been demonstrated to act synergistically with NGF in stimulating neurite outgrowth in PC12 cells as well as sensory ganglia; Lyons et al., PNAS, 91, pp. 3191-3195 (1994). This compound has also been shown to be neuroprotective in focal cerebral ischemia; J. Sharkey and S. P. Butcher, Nature, 371, pp.336-339 (1994) and to increase the rate of axonal regeneration in injured sciatic nerve, Gold et al., J. Neurosci., 15, pp. 7509-16 (1995).

Though a wide variety of neurological degenerative disorders may be treated by stimulating neurite outgrowth, there are relatively few agents known to possess these properties. Thus, there remains a great need for new pharmaceutically acceptable compounds and compositions that have the ability to ameliorating neuronal degeneration in patients.

It is a goal of the present invention to provide compositions and methods for preventing or ameliorating neuronal degeneration, e.g., for the abatement of these degenerative neurological processes.

The secreted inducing molecule, Shh, plays a role in neural induction and differentiation along the entire extent of the ventral neuraxis, from the spinal cord to the forebrain. The importance of Shh in normal patterning can be demonstrated in vitro where it is needed to induce spinal motor neurons (Roelink et al., 1994, 1995; Tanabe et al., 1995), serotonergic hindbrain neurons, dopaminergic midbrain neurons (Hynes et al., 1995a, b; Wang et al., 1995; Ye et al., 1998), and ventral forebrain cells (Ericson et al., 1995; Dale et al., 1997; Gunhaga et al., 2000). In vivo, mice with defects in Shh signaling are cyclopic (Chiang et al., 1996), while human embryos with Shh mutations develop holoprosencephaly, a loss of midline structures in the brain and face (Belloni et al., 1996; Roessler et al., 1996).

In addition to its role in early midline patterning, Shh is a mitogen for subsets of neural precursors, including those in the cerebellum (Wechsler-Reya and Scott, 1999), retina (Jensen and Wallace, 1997), and spinal cord (Kalyani et al., 1998). Experiments in which Shh was widely overexpressed in CNS precursor cells showed that many of these precursors, even dorsal ones, are competent to proliferate in response to Shh, but only for a time limited to the normal developmental period of neurogenesis (Rowitch et al. 1999). These experiments suggest that in different contexts, Shh may control cell differentiation or cell division.

The signaling pathway by which Shh mediates its effects is complex (reviewed in McMahon, 2000). In mammals, Shh can bind to two receptors, Ptc-1 and Ptc-2. In the absence of ligand, Ptc-1 inhibits the activity of Smoothened, which in turn leads to inhibition of Hedgehog target genes, including those encoding the transcription factors, Gli-1,2 and 3, and Ptc-1 itself. Hedgehog binding to Ptc-1, however, represses Ptc-1 activity, leading to derepression of Smoothened and activation of these target genes. A cell's expression of Ptc-1 appears to indicate an ability of that cell to respond to Shh.

Although Shh appears to be necessary for the induction of a number of ventral cells with specific neurotransmitter phenotypes, effects of Shh on postmitotic basal forebrain cholinergic neurons have not been studied previously. In addition to being candidates for hedgehog responsiveness, these neurons are thought to play a role in spatial memory formation, whereas their loss contributes to the cognitive and memory deficits in Alzheimer's disease (Gilmor et al., 1999; Lehericy et al.,1993). In previous studies we showed that Shh can have positive survival effects on dopaminergic and GABAergic neurons at later stages of development than the induction period (Miao et al., 1997). We wished to determine whether Shh might have similar effects on postmitotic cholinergic neurons. Such effects could indicate the possibility of a therapeutic role for Shh in Alzheimer's or other neurodegenerative diseases.

### SUMMARY OF THE INVENTION

In one aspect, this invention provides compositions for ameliorating neuronal degeneration comprising hedgehog therapeutics or patched (ptc)therapeutics in conjunction with neurotrophic factors. It has been observed that the hedgehog therapeutics provided herein increase the efficacy of the neurotrophic factors. For example, in one embodiment, the hedgehog therapeutic or the ptc therapeutic increases the potency of the neurotrophic factors by at least one order of magnitude and preferably two orders of magnitude. Numerous neurotrophic factors have been identified in the art and any of those factors may be utilized in the compositions of this invention. These neurotrophic factors include, but are not limited to, nerve growth factor (NGF), insulin growth factor (IGF-1) and its active truncated derivatives such as gIGF-1, acidic and basic fibroblast growth factor (aFGF and bFGF, respectively), platelet-derived growth factors (PDGF), brain-derived neurotrophic factor (BDNF), ciliary neurotrophic factors (CNTF), glial cell line-derived neurotrophic factor (GDNF), neurotrophin-3 (NT-3)and neurotrophin 4/5 (NT-4/5). The most preferred neurotrophic factor in the compositions of this invention is NGF.

In one embodiment, the conjoint therapy comprises administering compositions comprising neurotrophic factors in conjunction with various hedgehog or ptc therapeutics resulting in the increased efficacy by reducing the ED50 of the neurotrophic factors.

One aspect of the present application relates to a method for modulating survival and/or differentiation of neural cells by contacting cells, in vitro or in vivo with a hedgehog or ptc therapeutic in conjunction with a neurotrophic factor.

In one embodiment a method is provided for promoting the survival and/or differentiation of cholinergic neurons by contacting cells, in vitro or in vivo with a hedgehog therapeutic in conjunction with a neurotrophic factor. In another embodiment, the cholinergic neurons are neurons of the basal forebrain, and treatment is for Alzheimer's disease. In embodiments wherein the patient is treated with a ptc therapeutic, such therapeutics are preferably small organic molecules which mimic hedgehog effects on patched-mediated signals.

In other embodiments, the subject method can be used for protecting dopaminergic and/or GABAergic neurons of a mammal from neurodegeneration; for preventing or treating neurodegenerative disorder; for treatment of Parkinson's ; for treatment of Huntington's ; and/or for treatment of ALS. In embodiments wherein the patient is treated with a ptc therapeutic, such therapeutics are preferably small organic molecules which mimic hedgehog effects on patched-mediated signals.

Another aspect of the present application relates to a method for promoting the survival of neurons of the substantia nigra by contacting the cells, in vitro or in vivo, with compositions comprising a hedgehog therapeutic or ptc therapeutic and a neurotrophic factor, wherein the composition is administered in an amount effective for increasing the rate of survival of the neurons relative to the absence of administration of the compositions of the present invention.

Wherein the subject method is carried out using a hedgehog therapeutic, the hedgehog therapeutic preferably a polypeptide including a hedgehog portion comprising at least a bioactive extracellular portion of a hedgehog protein, e.g., the hedgehog portion includes at least 50, 100 or 150 amino acid residues of an N-terminal half of a hedgehog protein. In preferred embodiments, the hedgehog portion includes at least a portion of the hedgehog protein corresponding to a 19 kd fragment of the extracellular domain of a hedgehog protein.

In preferred embodiments, the hedgehog portion has an amino acid sequence at least 60, 75, 85, or 95 percent identical with a hedgehog protein of any of SEQ ID Nos. 10-18 or 20, though sequences identical to those sequence listing entries are also contemplated as useful in the present method. The hedgehog portion can be encoded by a nucleic acid which hybridizes under stringent conditions to a nucleic acid sequence of any of SEQ ID Nos. 1-9 or 19, e.g., the hedgehog portion can be encoded by a vertebrate hedgehog gene, especially a human hedgehog gene.

In certain embodiments, the hedgehog proteins of the present invention are modified by a lipophilic moiety or moieties at one or more internal sites of the mature, processed extracellular domain, and may or may not be also derivatized with lipophilic moieties at the N or C-terminal residues of the mature polypeptide. In other embodiments, the polypeptide is modified at the C-terminal residue with a hydrophobic moiety other than a sterol. In still other embodiments, the polypeptide is modified at the N-terminal residue with a cyclic (preferably polycyclic) lipophilic group. Various combinations of the above are also contemplated.

In other embodiments, the subject method can be carried out by administering a gene activation construct, wherein the gene activation construct is designed to recombine with a genomic hedgehog gene of the patient to provide a heterologous transcriptional regulatory sequence operatively linked to a coding sequence of the hedgehog gene.

In still other embodiments, the subject method can be practiced with the administration of a gene therapy construct encoding a hedgehog polypeptide. For instance, the gene therapy construct can be provided in a composition selected from a group consisting of a recombinant viral particle, a liposome, and a poly-cationic nucleic acid binding agent.

In certain embodiments, the polypeptide is purified to at least 80% by dry weight, and more preferably 90 or 95% by dry weight.

In other preferred embodiments, the isolated nucleic acid encodes a polypeptide having a hedgehog amino acid sequence encoded by a nucleic acid which hybridizes under stringent conditions to a nucleic acid sequence selected from the group consisting of SEQ ID Nos. 1-9 or 19, which hedgehog amino acid sequence of the polypeptide corresponds to a natural proteolytic product of a hedgehog protein. Such polypeptides preferably (i) binds to a patched protein, (ii) regulates differentiation of neuronal cells, (iii) regulates survival of differentiated neuronal cells, (iv) regulates proliferation of chondrocytes, (v) regulates proliferation of testicular germ line cells, and/or (vi) functionally replaces drosophila hedgehog in transgenic drosophila fly, or a combination thereof.

Still another aspect of the present invention provides a recombinant transfection system, e.g., such as may be useful for gene therapy, comprising (i) a gene construct including the coding sequence for a hedgehog polypeptide, operably linked to a transcriptional regulatory sequence for causing expression of the hedgehog polypeptide in eukaryotic cells, and (ii) a gene delivery composition for delivering said gene construct to a cell and causing the cell to be transfected with said gene construct. For instance, the gene delivery composition is selected from a group consisting of a recombinant viral particle, a liposome, and a poly-cationic nucleic acid binding agent.

Another aspect of the present invention provides a probe/primer comprising a substantially purified oligonucleotide, said oligonucleotide containing a region of nucleotide sequence which hybridizes under stringent conditions to at least 10 consecutive nucleotides of sense or antisense sequence to SEQ ID Nos. 1-9 or, 19, or naturally occurring mutants thereof. In preferred embodiments, the probe/primer includes a label group attached thereto and able to be detected. The present invention also provides a test kit for detecting cells which contain a hedgehog mRNA transcript, and includes such probe/primers.

Still another embodiment of the present invention provides a purified preparation of an antisense nucleic acid which specifically hybridizes to and inhibits expression of a gene encoding a hedgehog protein under physiological conditions, which nucleic acid is at least one of (i) a synthetic oligonucleotide, (ii) single-stranded, (iii) linear, (iv) 20 to 50 nucleotides in length, and (v) a DNA analog resistant to nuclease degradation.

### BRIEF DESCRIPTION OF THE DRAWINGS

FIG. 1. Immunostraining for ChAT and Ptc 1. Mice containing the LacZ insert driven off the Ptc 1 promoter are used to demonstrate co-localization of Ptc 1 (red) and ChAT (green) positive neurons. Many CHAT positive neurons are present at post-natal day 7 (A) in the medial septum when observed in cross section. There are many more Ptc 1 positive cells in this region (B) as well as a subset that label for both markers (C). This is also observed in the adult medial septum (D-F). There are again many cells that are co-localizing Ptc 1 and ChAT (F). Arrows mark cells expressing both markers.

FIG. 2. Cooperation of Shh and NGF. Cooperation can be measured by counting the number of CHAT immunopositive cells in cultures of E16 chlionergic neuron cultures (top). There is a large increase when Shh and NGF are together that is also observed in cultures derived from post-natal day 0 (bottom).

FIG. 3. Timecourse of ChAT Expression. The development of the cultures is examined at day 4, 8, 12, and 14. The cooperation of Shh and NGF can be observed by day 4 (red and green) with the more dramatic difference being seen by day 8 as compared with the control condition (blue).

FIG. 4. Total neurons in E16 Cultures. Cultures were scored for the neuronal marker NeuN. When Shh (4 ug/ml) or II-Shh (1 ug/ml) are present with NGF (50 ng/ml) there are more NeuN positive cells.

FIG. 5. BrdU Incorporation in Cholinergic Neuron Cultures. Incorporation of BrdU was used as a measure of proliferation in the cultures when added overnight at 2 days in vitro. Cultures grown in 10% serum had a statistically significant level of BrdU incorporation in the conditions that contained II-Shh 1 ug/ml and II-Shh plus NGF 50 ng/ml as compared with the control (top). When cultures were grown in a defined serum free medium (bottom) there was still a statistically significant level of BRdU incorporation in the conditions containing Shh4 ug/ml.

FIG. 6. Thymidine Incorporation at Day 2. After incubation with tritiated thymidine the cultures are left to grow for a total of eight days in vitro. All ChAT positive cells are labeled with a dark brown reaction product (arrows). Nuclei heavily labeled for thymidine appear covered with black silver grains (arrow heads). There are no cells that double label for ChAT and thymidine incorporation. The combination of Shh and NGF together (E, F) have not influenced the precursors to divide yielding double labeled cells as compared with the controls (A-D).

FIG. 7. Thymidine Incorporation at Day 6. After incubation with tritiated thymidine the cultures are left to grow for a total of eight days in vitro. All ChAT positive cells are labeled with a dark brown reaction product (arrows). Nuclei heavily labeled for thymidine appear covered with silver grains (arrow heads). There are no cells that double label for ChAT and thymidine incorporation. The combination of Shh and NGF (E, F) have not influenced the ChAT precursors to divide yielding double labeled cells as compared with the controls (A-D).

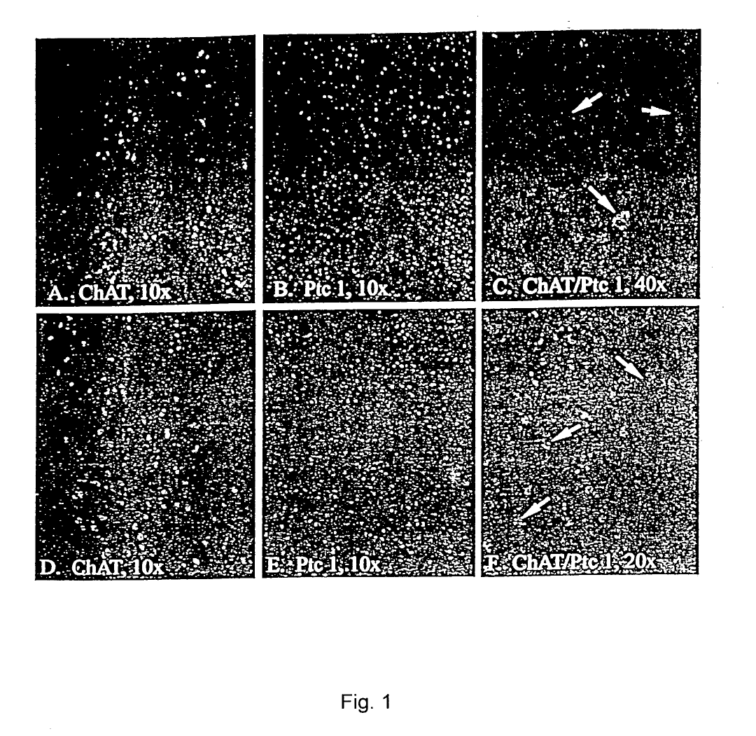

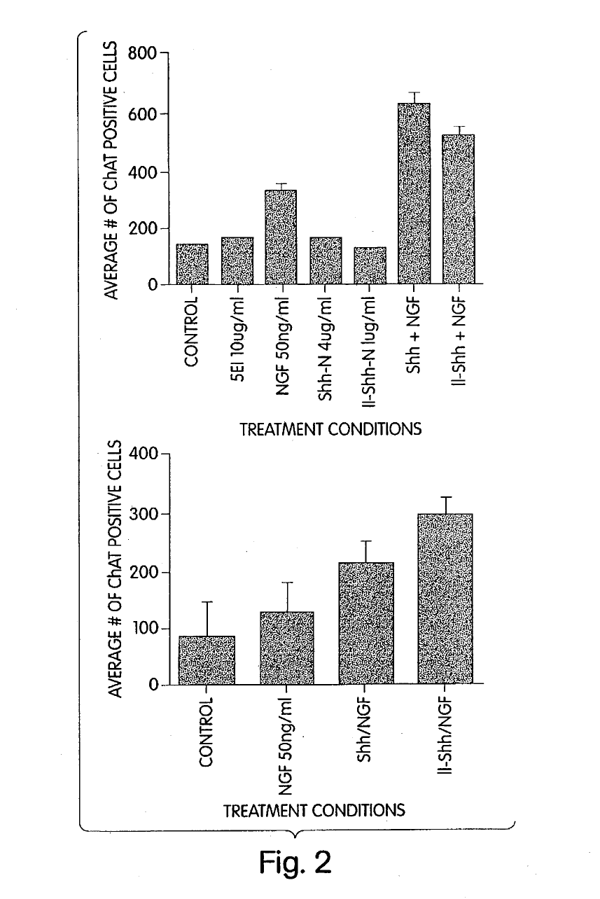

### DETAILED DESCRIPTION OF THE INVENTION

I. General

Neurotrophic factors are known to promote survival and differentiation of a wide variety of neurons and other brain cells. This invention pertains to the joint treatment, in vitro or in vivo, of neuronal cells with compositions comprising neurotrophic factors and hedgehog or ptc therapeutics. The hedgehog therapeutics include but are not limited to the hedgehog polypeptides set forth in SEQ ID Nos: 10-18 and 20 and fragments thereof. The neurotrophic factors include but are not limited to These neurotrophic factors include, but are not limited to, nerve growth factor (NGF), insulin growth factor (IGF-1) and its active truncated derivatives such as gIGF-1, acidic and basic fibroblast growth factor (aFGF and bFGF, respectively), platelet-derived growth factors (PDGF), brain-derived neurotrophic factor (BDNF), ciliary neurotrophic factors (CNTF), glial cell line-derived neurotrophic factor (GDNF), neurotrophin-3 (NT-3)and neurotrophin 4/5 (NT-4/5). In a preferred embodiment the composition comprises a hedgehog therapeutic in combinatioin with NGF.

In one embodiment a method is provided for promoting the survival and/or differentiation of cholinergic neurons by contacting cells, in vitro or in vivo with a hedgehog therapeutic in conjunction with a neurotrophic factor. In another embodiment, the cholinergic neurons are neurons of the basal forebrain, and treatment is for Alzheimer's disease. In embodiments wherein the patient is treated with a ptc therapeutic, such therapeutics are preferably small organic molecules which mimic hedgehog effects on patched-mediated signals.

In other embodiments, the subject method can be used for protecting dopaminergic and/or GABAergic neurons of a mammal from neurodegeneration; for preventing or treating neurodegenerative disorder; for treatment of Parkinson's ; for treatment of Huntington's ; and/or for treatment of ALS. In embodiments wherein the patient is treated with a ptc therapeutic, such therapeutics are preferably small organic molecules which mimic hedgehog effects on patched-mediated signals.

Sonic hedgehog (Shh), an axis-determining secreted protein, is expressed during early vertebrate embryogenesis in the notochord and ventral neural tube. In this site it plays a role in the phenotypic specification of ventral neurons along the length of the CNS. For example, Shh induces the differentiation of motor neurons in the spinal cord and dopaminergic neurons in the midbrain.

We have determined that hedgehog and ptc therapeutics lower the ED50 for neurotrophic factors to affect differentiation and/or survival of neuronal cells. In other words, the hedgehog therapeutics increase the efficacy and/or potency of the neurotrophic factors. Based in part on these findings, we have determined that this combination of agents is useful as protective agents in the treatment and prophylaxis for neurodegenerative disorders, including, without limitation, Alzheimer's disease, Parkinson's disease, Huntington's disease, amyotrophic lateral sclerosis and the like.

The compositions described herein may further include hedgehog or hedgehog agonists as cell culture additives for the maintenance and differentiation of neurons in cultures, e.g., in cultures of cholinergic neurons. The subject methods and compositions can also be used to augment the implantion of such neuronal cells in an animal.

II. Definitions

For convience, certain terms employed in the specfication, examples, and appended claims are collected here.

The term “hedgehog therapeutic” refers to various forms of hedgehog polypeptides, as well as peptidomimetics, which are neuroprotective for neuronal cells, and in particular, enhance the survival of cholinergic, dopaminergic and/or GABA-ergic neurons. These include naturally occurring forms of hedgehog polypeptides, as well as modified or mutant forms generated by molecular biological techniques, chemical synthesis, etc. While in preferred embodiments the hedgehog polypeptide is derived from a vertebrate homolog, cross-sepcies activity reported in the literature supports the use of hedgehog peolypeptides from invertebrate organisms as well. Naturally and non-naturally occurring hedgehog therapeutics referred to herein as “agonists” mimic or potentiate (collectively “agonize”) the effects of a naturally-occurring hedgehog polypeptide as a neuroprotective agent. In addition, the term “hedgehog therapeutic” includes molecules which can activate expression of an endogenous hedgehog gene. The term also includes gene therapy constructs for causing expression of hedgehog polypeptides in vivo, as for example, expression constructs encoding recombinant hedgehog polypeptides as well as trans-activation constructs for altering the regulatory sequences of an endogenous hedgehog gene by homologous recombination.

In particular, the term “hedgehog polypeptide” encompasses hedgehog polypeptides and peptidyl fragments thereof.

As used herein the term “bioactive fragment”, with reference to a portions of hedgehog polypeptides, refers to a fragment of a full-length hedgehog polypeptide, wherein the fragment specifically agonizes neuroprotective events mediated by wild-type hedgehog polypeptides. The hedgehog bioactive fragment preferably is a soluble extracellular portion of a hedgehog polypeptide, where solubility is with reference to physiologically compatible solutions. Exemplary bioactive fragments are described in PCT publications WO 95/18856 and WO 96/17924.

The term “ptc therapeutic” refers to agents which mimic the effect of naturally occurring hedgehog polypeptides on patched signaling. The ptc therapeutic can be, e.g., a peptide, a nucleic acid, a carbohydrate, a small organic molecule, or natural product extract (or fraction thereof).

As used herein, the term “antibody homolog” includes intact antibodies consisting of immunoglobulin light and heavy chains linked via disulfide bonds. The term “antibody homolog” is also intended to encompass a protein comprising one or more polypeptides selected from immunoglobulin heavy chains and antigen-binding fragments thereof which are capable of binding to one or more antigens (i.e., hedgehog or patched). The component polypeptides of an antibody homolog composed of more than one polypeptide may optionally be disulfide-bound or otherwise covalently crosslinked. Accordingly, therefore, “antibody homologs” include intact immunoglobulins of types IgA, IgG, IgE, IgD, IgM (as well as subtypes thereof), wherein the light chains of the immunoglobulin may be of types kappa or lambda. Preferred proteins of the invention may include portions of intact antibodies that retain antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like.

As used herein, a “humanized antibody homolog” is an antibody homolog, produced by recombinant DNA technology, in which some or all of the amino acids of a human immunoglobulin light or heavy chain that are not required for antigen binding have been substituted for the corresponding amino acids from a nonhuman mammalian immunoglobulin light or heavy chain. A “human antibody homolog” is an antibody homolog in which all the amino acids of an immunoglobulin light or heavy chain (regardless of whether or not they are required for antigen binding) are derived from a human source.

A “patient” or “subject” to be treated by the subject method are mammals, including humans.

An “effective amount” of, e.g., a hedgehog or ptc therapeutic, with respect to the subject method of treatment, refers to an amount of the therapeutic in a preparation which, when applied as part of a desired dosage regimen causes a increase in survival of a neuronal cell population according to clinically acceptable standards for the treatment or prevention of a particular disorder.

By “prevent degeneration” it is meant reduction in the loss of cells (such as from apoptosis), or reduction in impairment of cell function, e.g., release of dopamine in the case of dopaminergic neurons.

A “trophic amount” of the composition comprising a hedgehog or ptc therapeutic in combination with a neurotrophic factor is an amount sufficient to, under the circumstances, cause an increase in the rate of survival or the functional performance of a hedgehog-responsive cell, e.g., a dopaminergic, cholinergic and/or GABAergic cell.

“Homology” and “identity” each refer to sequence similarity between two polypeptide sequences, with identity being a more strict comparison. Homology and identity can each be determined by comparing a position in each sequence which may be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. An “unrelated” or “non-homologous” sequence shares less than 40 percent identity, though preferably less than 25 percent identity, with an hedgehog sequence.

In particular, the term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. Various alignment algorithms and/or programs may be used, including FASTA, BLAST or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

The term “corresponds to”, when referring to a particular polypeptide or nucleic acid sequence is meant to indicate that the sequence of interest is identical or homologous to the reference sequence to which it is said to correspond.

The terms “recombinant protein”, “heterologous protein” and “exogenous protein” are used interchangeably throughout the specification and refer to a polypeptide which is produced by recombinant DNA techniques, wherein generally, DNA encoding the polypeptide is inserted into a suitable expression construct which is in turn used to transform a host cell to produce the heterologous protein. That is, the polypeptide is expressed from a heterologous nucleic acid.

A “chimeric protein” or “fusion protein” is a fusion of a first amino acid sequence encoding a hedgehog polypeptide with a second amino acid sequence defining a domain foreign to and not substantially homologous with any domain of hh protein. A chimeric protein may present a foreign domain which is found (albeit in a different protein) in an organism which also expresses the first protein, or it may be an “interspecies”, “intergenic”, etc. fusion of protein structures expressed by different kinds of organisms. In general, a fusion protein can be represented by the general formula (X)ₙ−(hh)ₘ−(Y)ₙ, wherein hh represents all or a portion of the hedgehog polypeptide, X and Y each independently represent an amino acid sequences which are not naturally found as a polypeptide chain contiguous with the hedgehog sequence, m is an integer greater than or equal to 1, and each occurrence of n is, independently, 0 or an integer greater than or equal to 1 (n and m are preferably no greater than 5 or 10).

As used herein, the term “vector” refers to a nucleic acid molecule capable of transporting another nucleic acid to which it has been linked. The term “expression vector” includes plasmids, cosmids or phages capable of synthesizing, for example, the subject hedgehog polypeptides encoded by the respective recombinant gene carried by the vector. Preferred vectors are those capable of autonomous replication and/expression of nucleic acids to which they are linked. In the present specification, “plasmid” and “vector” are used interchangeably as the plasmid is the most commonly used form of vector. Moreover, the invention is intended to include such other forms of expression vectors which serve equivalent functions and which become known in the art subsequently hereto.

“Transcriptional regulatory sequence” is a generic term used throughout the specification to refer to DNA sequences, such as initiation signals, enhancers, and promoters, as well as polyadenylation sites, which induce or control transcription of protein (or antisense) coding sequences with which they are operably linked. In preferred embodiments, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the regulatory protein.

The term “operably linked” refers to the arrangement of a transcriptional regulatory element relative to other transcribable nucleic acid sequence such that the transcriptional regulatory element can regulate the rate of transcription from the transcribable sequence(s).

III. Exemplary Hedgehog Therapeutic Compounds

The hedgehog therapeutic compositions of the subject method can be generated by any of a variety of techniques, including purification of naturally occurring proteins, recombinantly produced proteins and synthetic chemistry. Polypeptide forms of the hedgehog therapeutics are preferably derived from vertebrate hedgehog proteins, e.g., have sequences corresponding to naturally occurring hedgehog proteins, or fragments thereof, from vertebrate organisms. However, it will be appreciated that the hedgehog polypeptide can correspond to a hedgehog protein (or fragment thereof) which occurs in any metazoan organism.

The various naturally-occurring hedgehog proteins from which the subject therapeutics can be derived are characterized by a signal peptide, a highly conserved N-terminal region, and a more divergent C-terminal domain. In addition to signal sequence cleavage in the secretory pathway (Lee, J. J. et al. (1 992) Cell 71:33-50; Tabata, T. et al. (1992) Genes Dev. 2635-2645; Chang, D. E. et al. (1994) Development 120:3339-3353), hedgehog precursor proteins naturally undergo an internal autoproteolytic cleavage which depends on conserved sequences in the C-terminal portion (Lee et al. (1994) Science 266:1528-1537; Porter et al. (1995) Nature 374:363-366). This autocleavage leads to a 19 kD N-terminal peptide and a C-terminal peptide of 26-28 kD (Lee et al. (1992) supra; Tabata et al. (1992) supra; Chang et al. (1994) supra; Lee et al. (1994) supra; Bumcrot, D. A., et al. (1995) Mol. Cell. Biol. 15:2294-2303; Porter et al. (1995) supra; Ekker, S. C. et al. (1995) Curr. Biol. 5:944-955; Lai, C. J. et al. (1995) Development 121:2349-2360). The N-terminal peptide stays tightly associated with the surface of cells in which it was synthesized, while the C-terminal peptide is freely diffusible both in vitro and in vivo (Lee et al. (1994) supra; Bumcrot et al. (1995) supra; Mart', E. et al. (1995) Development 121:2537-2547; Roelink, H. et al. (1995) Cell 81:445-455). Cell surface retention of the N-terminal peptide is dependent on autocleavage, as a truncated form of hedgehog encoded by an RNA which terminates precisely at the normal position of internal cleavage is diffusible in vitro (Porter et al. (1995) supra) and in vivo (Porter, J. A. et al. (1996) Cell 86, 21-34). Biochemical studies have shown that the autoproteolytic cleavage of the hedgehog precursor protein proceeds through an internal thioester intermediate which subsequently is cleaved in a nucleophilic substitution. It is suggested that the nucleophile is a small lipophilic molecule, more particularly cholesterol, which becomes covalently bound to the C-terminal end of the N-peptide (Porter et al. (1996) supra), tethering it to the cell surface.

The vertebrate family of hedgehog genes includes at least four members, e.g., paralogs of the single drosophila hedgehog gene (SEQ ID No. 19). Three of these members, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears specific to fish. According to the appended sequence listing, (see also Table 1) a chicken Shh polypeptide is encoded by SEQ ID No: 1; a mouse Dhh polypeptide is encoded by SEQ ID No: 2; a mouse Ihh polypeptide is encoded by SEQ ID No: 3; a mouse Shh polypeptide is encoded by SEQ ID No: 4 a zebrafish Shh polypeptide is encoded by SEQ ID No: 5; a human Shh polypeptide is encoded by SEQ ID No: 6; a human Ihh polypeptide is encoded by SEQ ID No: 7; a human Dhh polypeptide is encoded by SEQ ID No. 8; and a zebrafish Thh is encoded by SEQ ID No. 9.

TABLE 1

| Guide to hedgehog | | |
|---|---|---|
| sequences in | | |
| Sequence Listing | Nucleotide | Amino Acid |
| Chicken Shh | SEQ ID No.1 | SEQ ID No.10 |
| Mouse Dhh | SEQ ID No.2 | SEQ ID No.11 |
| Mouse Ihh | SEQ ID No.3 | SEQ ID No.12 |
| Mouse Shh | SEQ ID No.4 | SEQ ID No.13 |
| Zebrafish Shh | SEQ ID No.5 | SEQ ID No.14 |
| Human Shh | SEQ ID No.6 | SEQ ID No.15 |
| Human Ihh | SEQ ID No.7 | SEQ ID No.16 |
| Human Dhh | SEQ ID No.8 | SEQ ID No.17 |
| Zebrafish Thh | SEQ ID No.9 | SEQ ID No.18 |
| Drosophila HH | SEQ ID No.19 | SEQ ID No.20 |

In addition to the sequence variation between the various hedgehog homologs, the hedgehog proteins are apparently present naturally in a number of different forms, including a pro-form, a full-length mature form, and several processed fragments thereof. The pro-form includes an N-terminal signal peptide for directed secretion of the extracellular domain, while the full-length mature form lacks this signal sequence.

As described above, further processing of the mature form occurs in some instances to yield biologically active fragments of the protein. For instance, sonic hedgehog undergoes additional proteolytic processing to yield two peptides of approximately 19 kDa and 27 kDa, the 19 kDa fragment corresponding to an proteolytic N-terminal portion of the mature protein.

In addition to proteolytic fragmentation, the vertebrate hedgehog proteins can also be modified post-translationally, such as by glycosylation and/or addition of lipophilic moieties, such as stents, fatty acids, etc., though bacterially produced (e.g. unmodified) forms of the proteins still maintain certain of the bioactivities of the native protein. Bioactive fragments of hedgehog polypeptides of the present invention have been generated and are described in great detail in, e.g., PCT publications WO 95/18856 and WO 96/17924.

There are a wide range of lipophilic moieties with which hedgehog polypeptides can be derivatived. The term “lipophilic group”, in the context of being attached to a hedgehog polypeptide, refers to a group having high hydrocarbon content thereby giving the group high affinity to lipid phases. A lipophilic group can be, for example, a relatively long chain alkyl or cycloalkyl (preferably n-alkyl) group having approximately 7 to 30 carbons. The alkyl group may terminate with a hydroxy or primary amine “tail”. To further illustrate, lipophilic molecules include naturally-occurring and synthetic aromatic and non-aromatic moieties such as fatty acids, sterols, esters and alcohols, other lipid molecules, cage structures such as adamantane and buckminsterfullerenes, and aromatic hydrocarbons such as benzene, perylene, phenanthrene, anthracene, naphthalene, pyrene, chrysene, and naphthacene.

In one embodiment, the hedgehog polypeptide is modified with one or more sterol moieties, such as cholesterol. See, for example, PCT publication WO 96/17924. In certain embodiments, the cholesterol is preferably added to the C-terminal glycine were the hedgehog polypeptide corresponds to the naturally-occurring N-terminal proteolytic fragment.

In another embodiment, the hedgehog polypeptide can be modified with a fatty acid moiety, such as a myrostoyl, palmitoyl, stearoyl, or arachidoyl moiety. See, e.g., Pepinsky et al. (1998) J Biol. Chem 273: 14037.

In addition to those effects seen by cholesterol-addition to the C-terminus or fatty acid addition to the N-terminus of extracellular fragments of the protein, at least certain of the biological activities of the hedgehog gene products are unexpectedly potentiated by derivativation of the protein with lipophilic moieties at other sites on the protein and/or by moieties other than cholesterol or fatty acids. Certain aspects of the invention are directed to the use of preparations of hedgehog polypeptides which are modified at sites other than N-terminal or C-terminal residues of the natural processed form of the protein, and/or which are modified at such terminal residues with lipophilic moieties other than a sterol at the C-terminus or fatty acid at the N-terminus.

Particularly useful as lipophilic molecules are alicyclic hydrocarbons, saturated and unsaturated fatty acids and other lipid and phospholipid moieties, waxes, cholesterol, isoprenoids, terpenes and polyalicyclic hydrocarbons including adamantane and buckminsterfullerenes, vitamins, polyethylene glycol or oligoethylene glycol, (C1-C18)-alkyl phosphate diesters, —O—CH2-CH(OH)—O—(C12-C18)-alkyl, and in particular conjugates with pyrene derivatives. The lipophilic moiety can be a lipophilic dye suitable for use in the invention include, but are not limited to, diphenylhexatriene, Nile Red, N-phenyl-1-naphthylamine, Prodan, Laurodan, Pyrene, Perylene, rhodamine, rhodamine B, tetramethylrhodamine, Texas Red, sulforhodamine, 1,1′-didodecyl-3,3,3′,3′tetramethylindocarbocyanine perchlorate, octadecyl rhodamine B and the BODIPY dyes available from Molecular Probes Inc.

Other exemplary lipophilic moietites include aliphatic carbonyl radical groups include 1- or 2-adamantylacetyl, 3-methyladamant-1-ylacetyl, 3-methyl-3-bromo-1-adamantylacetyl, 1-decalinacetyl, camphoracetyl, camphaneacetyl, noradamantylacetyl, norbornaneacetyl, bicyclo[2.2.2.]-oct-5-eneacetyl, 1-methoxybicyclo[2.2.2.]-oct-5-ene-2-carbonyl, cis-5-norbornene-endo-2,3-dicarbonyl, 5-norbornen-2-ylacetyl, (1R)-(−)-myrtentaneacetyl, 2-norbornaneacetyl, anti-3-oxo-tricyclo[2.2.1.0<2,6>]-heptane-7-carbonyl, decanoyl, dodecanoyl, dodecenoyl, tetradecadienoyl, decynoyl or dodecynoyl.

The hedgehog polypeptide can be linked to the hydrophobic moiety in a number of ways including by chemical coupling means, or by genetic engineering.

There are a large number of chemical cross-linking agents that are known to those skilled in the art. For the present invention, the preferred cross-linking agents are heterobifunctional cross-linkers, which can be used to link the hedgehog polypeptide and hydrophobic moiety in a stepwise manner. Heterobifunctional cross-linkers provide the ability to design more specific coupling methods for conjugating to proteins, thereby reducing the occurrences of unwanted side reactions such as homo-protein polymers. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC—SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl (DMP) are examples of useful homobifunctional cross-linking agents, and bis-[β-(4-azidosalicylamido)ethyl]disulfide (BASED) and N-succinimidyl-6(4′-azido-2′-nitrophenyl-amino)hexanoate (SANPAH) are examples of useful photoreactive cross-linkers for use in this invention. For a recent review of protein coupling techniques, see Means et al. (1990) Bioconjugate Chemistry 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's . Both of these reactive groups result in the formation of stable thioether bonds.

The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules. For instance, SMPB has a span of 14.5 angstroms.

Preparing protein-protein conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using for instance, N-ethylmaleimide (see Partis et al. (1983) J. Pro. Chem. 2:263, incorporated by reference herein). Ellman's Reagent can be used to calculate the quantity of sulfhydryls in a particular protein (see for example Ellman et al. (1958) Arch. Biochem. Biophys. 74:443 and Riddles et al. (1979) Anal. Biochem. 94:75, incorporated by reference herein).

The reaction buffer should be free of extraneous amines and sulfhydryls. The pH of the reaction buffer should be 7.0-7.5. This pH range prevents maleimide groups from reacting with amines, preserving the maleimide group for the second reaction with sulfhydryls.

The NHS-ester containing cross-linkers have limited water solubility. They should be dissolved in a minimal amount of organic solvent (DMF or DMSO) before introducing the cross-linker into the reaction mixture. The cross-linker/solvent forms an emulsion which will allow the reaction to occur.

The sulfo-NHS ester analogs are more water soluble, and can be added directly to the reaction buffer. Buffers of high ionic strength should be avoided, as they have a tendency to “salt out” the sulfo-NHS esters. To avoid loss of reactivity due to hydrolysis, the cross-linker is added to the reaction mixture immediately after dissolving the protein solution.

The reactions can be more efficient in concentrated protein solutions. The more alkaline the pH of the reaction mixture, the faster the rate of reaction. The rate of hydrolysis of the NHS and sulfo-NHS esters will also increase with increasing pH. Higher temperatures will increase the reaction rates for both hydrolysis and acylation.

Once the reaction is completed, the first protein is now activated, with a sulfhydryl reactive moiety. The activated protein may be isolated from the reaction mixture by simple gel filtration or dialysis. To carry out the second step of the cross-linking, the sulfhydryl reaction, the lipophilic group chosen for reaction with maleimides, activated halogens, or pyridyl disulfides must contain a free sulfhydryl. Alternatively, a primary amine may be modified with to add a sulfhydryl

In all cases, the buffer should be degassed to prevent oxidation of sulfhydryl groups. EDTA may be added to chelate any oxidizing metals that may be present in the buffer. Buffers should be free of any sulfhydryl containing compounds.

Maleimides react specifically with -SH groups at slightly acidic to neutral pH ranges (6.5-7.5). A neutral pH is sufficient for reactions involving halogens and pyridyl disulfides. Under these conditions, maleimides generally react with —SH groups within a matter of minutes. Longer reaction times are required for halogens and pyridyl disulfides.

The first sulfhydryl reactive-protein prepared in the amine reaction step is mixed with the sulfhydryl-containing lipophilic group under the appropriate buffer conditions. The conjugates can be isolated from the reaction mixture by methods such as gel filtration or by dialysis.

Exemplary activated lipophilic moieties for conjugation include: N-(1-pyrene)maleimide; 2,5-dimethoxystilbene-4′-maleimide, eosin-5-maleimide; fluorescein-5-maleimide; N-(4-(6-dimethylamino-2-benzofuranyl)phenyl)maleimide; benzophenone-4-maleimide; 4-dimethylaminophenylazophenyl-4′-maleimide (DABMI), tetramethylrhodamine-5-maleimide, tetramethylrhodamine-6-maleimide, Rhodamine Red™ C2 maleimide, N-(5-aminopentyl)maleimide, trifluoroacetic acid salt, N-(2-aminoethyl)maleimide, trifluoroacetic acid salt, Oregon Green™ 488 maleimide, N-(2-((2-(((4-azido-2,3,5,6-tetrafluoro)benzoyl) amino)ethyl)dithio)ethyl)maleimide (TFPAM-SS1), 2-(1-(3-dimethylaminopropyl)-indol-3-yl)-3-(indol-3-yl) maleimide (bisindolylmaleimide; GF 109203X), BODIPY® FL N-(2-aminoethyl)maleimide, N-(7-dimethylamino-4-methylcoumarin-3-yl)maleimide (DACM), Alexa™ 488 C5 maleimide, Alexa™ 594 C5 maleimide, sodium saltN-(1-pyrene)maleimide, 2,5-dimethoxystilbene-4′-maleimide, eosin-5-maleimide, fluorescein-5-maleimide, N-(4-(6-dimethylamino-2-benzofuranyl)phenyl)maleimide, benzophenone-4-maleimide, 4-dimethylaminophenylazophenyl-4′-maleimide, 1-(2-maleimidylethyl)-4-(5-(4-methoxyphenyl)oxazol-2-yl)pyridinium methanesulfonate, tetramethylrhodamine-5-maleimide, tetramethylrhodamine-6-maleimide, Rhodamine Red™ C2 maleimide, N-(5-aminopentyl)maleimide, N-(2-aminoethyl)maleimide, N-(2-((2-(((4-azido-2,3,5,6-tetrafluoro)benzoyl) amino)ethyl)dithio)ethyl)maleimide, 2-(1-(3-dimethylaminopropyl)-indol-3-yl)-3-(indol-3-yl) maleimide, N-(7-dimethylamino-4-methylcoumarin-3-yl)maleimide (DACM), 11H-Benzo[a]fluorene, Benzo[a]pyrene.

In one embodiment, the hedgehog polypeptide can be derivatived using pyrene maleimide, which can be purchased from Molecular Probes (Eugene, Oreg.), e.g., N-(1-pyrene)maleimide or 1-pyrenemethyl iodoacetate (PMIA ester).

For those embodiments wherein the hydophobic moiety is a polypeptide, the modified hedgehog polypeptide of this invention can be constructed as a fusion protein, containing the hedgehog polypeptide and the hydrophobic moiety as one contiguous polypeptide chain.

In certain embodiments, the lipophilic moiety is an amphipathic polypeptide, such as magainin, cecropin, attacin, melittin, gramicidin S, alpha-toxin of Staph. aureus, alamethicin or a synthetic amphipathic polypeptide. Fusogenic coat proteins from viral particles can also be a convenient source of amphipathic sequences for the subject hedgehog proteins Moreover, mutagenesis can be used to create modified hh polypeptides, e.g., for such purposes as enhancing therapeutic or prophylactic efficacy, or stability (e.g., ex vivo shelf life and resistance to proteolytic degradation in vivo). Such modified peptides can be produced, for instance, by amino acid substitution, deletion, or addition. Modified hedgehog polypeptides can also include those with altered post-translational processing relative to a naturally occurring hedgehog protein, e.g., altered glycosylation, cholesterolization, prenylation and the like.

In one embodiment, the hedgehog therapeutic is a polypeptide encodable by a nucleotide sequence that hybridizes under stringent conditions to a hedgehog coding sequence represented in one or more of SEQ ID Nos: 1-7. Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

As described in the literature, genes for other hedgehog proteins, e.g., from other animals, can be obtained from mRNA or genomic DNA samples using techniques well known in the art. For example, a cDNA encoding a hedgehog protein can be obtained by isolating total mRNA from a cell, e.g. a mammalian cell, e.g. a human cell, including embryonic cells. Double stranded cDNAs can then be prepared from the total mRNA, and subsequently inserted into a suitable plasmid or bacteriophage vector using any one of a number of known techniques. The gene encoding a hedgehog protein can also be cloned using established polymerase chain reaction techniques.

Preferred nucleic acids encode a hedgehog polypeptide comprising an amino acid sequence at least 60% homologous or identical, more preferably 70% homologous or identical, and most preferably 80% homologous or identical with an amino acid sequence selected from the group consisting of SEQ ID Nos: 8-14. Nucleic acids which encode polypeptides at least about 90%, more preferably at least about 95%, and most preferably at least about 98-99% homology or identity with an amino acid sequence represented in one of SEQ ID Nos: 8-14 are also within the scope of the invention.

In addition to native hedgehog proteins, hedgehog polypeptides preferred by the present invention are at least 60% homologous or identical, more preferably 70% homologous or identical and most preferably 80% homologous or identical with an amino acid sequence represented by any of SEQ ID Nos: 8-14. Polypeptides which are at least 90%, more preferably at least 95%, and most preferably at least about 98-99% homologous or identical with a sequence selected from the group consisting of SEQ ID Nos: 8-14 are also within the scope of the invention. The only prerequisite is that the hedgehog polypeptide is capable of modulating the growth state of peripheral nerve cells.

The term “recombinant protein” refers to a polypeptide of the present invention which is produced by recombinant DNA techniques, wherein generally, DNA encoding a hedgehog polypeptide is inserted into a suitable expression vector which is in turn used to transform a host cell to produce the heterologous protein. Moreover, the phrase “derived from”, with respect to a recombinant hedgehog gene, is meant to include within the meaning of “recombinant protein” those proteins having an amino acid sequence of a native hedgehog protein, or an amino acid sequence similar thereto which is generated by mutations including substitutions and deletions (including truncation) of a naturally occurring form of the protein.

The method of the present invention can also be carried out using variant forms of the naturally occurring hedgehog polypeptides, e.g., mutational variants.

As is known in the art, hedgehog polypeptides can be produced by standard biological techniques or by chemical synthesis. For example, a host cell transfected with a nucleic acid vector directing expression of a nucleotide sequence encoding the subject polypeptides can be cultured under appropriate conditions to allow expression of the peptide to occur. The polypeptide hedgehog may be secreted and isolated from a mixture of cells and medium containing the recombinant hedgehog polypeptide. Alternatively, the peptide may be retained cytoplasmically by removing the signal peptide sequence from the recombinant hedgehog gene and the cells harvested, lysed and the protein isolated. A cell culture includes host cells, media and other byproducts. Suitable media for cell culture are well known in the art. The recombinant hedgehog polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant hedgehog polypeptide is a fusion protein containing a domain which facilitates its purification, such as an hedgehog/GST fusion protein. The host cell may be any prokaryotic or eukaryotic cell.

Recombinant hedgehog genes can be produced by ligating nucleic acid encoding an hedgehog protein, or a portion thereof, into a vector suitable for expression in either prokaryotic cells, eukaryotic cells, or both. Expression vectors for production of recombinant forms of the subject hedgehog polypeptides include plasmids and other vectors. For instance, suitable vectors for the expression of a hedgehog polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due to the presence of the pBR322 ori, and in S. cerevisiae due to the replication determinant of the yeast 2 micron plasmid. In addition, drug resistance markers such as ampicillin can be used. In an illustrative embodiment, an hedgehog polypeptide is produced recombinantly utilizing an expression vector generated by sub-cloning the coding sequence of one of the hedgehog genes represented in SEQ ID Nos: 1-7.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press: 1989) Chapters 16 and 17.

In some instances, it may be desirable to express the recombinant hedgehog polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

When it is desirable to express only a portion of an hedgehog protein, such as a form lacking a portion of the N-terminus, i.e. a truncation mutant which lacks the signal peptide, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing hedgehog-derived polypeptides in a host which produces MAP (e.g., E. coli or CM89 or S. cerevisiae), or in vitro by use of purified MAP (e.g., procedure of Miller et al., supra).

Alternatively, the coding sequences for the polypeptide can be incorporated as a part of a fusion gene including a nucleotide sequence encoding a different polypeptide. It is widely appreciated that fusion proteins can also facilitate the expression of proteins, and accordingly, can be used in the expression of the hedgehog polypeptides of the present invention. For example, hedgehog polypeptides can be generated as glutathione-S-transferase (GST-fusion) proteins. Such GST-fusion proteins can enable easy purification of the hedgehog polypeptide, as for example by the use of glutathione-derivatized matrices (see, for example, Current Protocols in Molecular Biology, eds. Ausubel et al. (N.Y.: John Wiley & Sons, 1991)). In another embodiment, a fusion gene coding for a purification leader sequence, such as a poly-(His)/enterokinase cleavage site sequence, can be used to replace the signal sequence which naturally occurs at the N-terminus of the hedgehog protein (e.g.of the pro-form, in order to permit purification of the poly(His)-hedgehog protein by affinity chromatography using a Ni²⁺ metal resin. The purification leader sequence can then be subsequently removed by treatment with enterokinase (e.g., see Hochuli et al. (1987) J. Chromatography 411:177; and Janknecht et al. PNAS 88:8972).

Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

Hedgehog polypeptides may also be chemically modified to create hedgehog derivatives by forming covalent or aggregate conjugates with other chemical moieties, such as glycosyl groups, cholesterol, isoprenoids, lipids, phosphate, acetyl groups and the like. Covalent derivatives of hedgehog proteins can be prepared by linking the chemical moieties to functional groups on amino acid sidechains of the protein or at the N-terminus or at the C-terminus of the polypeptide.

For instance, hedgehog proteins can be generated to include a moiety, other than sequence naturally associated with the protein, that binds a component of the extracellular matrix and enhances localization of the analog to cell surfaces. For example, sequences derived from the fibronectin “type-III repeat”, such as a tetrapeptide sequence R-G-D-S (Pierschbacher et al. (1984) Nature 309:30-3; and Kornblihtt et al. (1985) EMBO 4:1755-9) can be added to the hedgehog polypeptide to support attachment of the chimeric molecule to a cell through binding ECM components (Ruoslahti et al. (1987) Science 238:491-497; Pierschbacheret al. (1987) J. Biol. Chem. 262:17294-8.; Hynes (1987) Cell 48:549-54; and Hynes (1992) Cell 69:11-25).

In a preferred embodiment, the hedgehog polypeptide is isolated from, or is otherwise substantially free of, other cellular proteins, especially other extracellular or cell surface associated proteins which may normally be associated with the hedgehog polypeptide, unless provided in the form of fusion protein with the hedgehog polypeptide. The term “substantially free of other cellular or extracellular proteins” (also referred to herein as “contaminating proteins”) or “substantially pure preparations” or “purified preparations” are defined as encompassing preparations of hedgehog polypeptides having less than 20% (by dry weight) contaminating protein, and preferably having less than 5% contaminating protein. By “purified”, it is meant that the indicated molecule is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

As described above for recombinant polypeptides, isolated hedgehog polypeptides can include all or a portion of the amino acid sequences represented in any of SEQ ID Nos: 10-18 or 20, or a homologous sequence thereto. Preferred fragments of the subject hedgehog proteins correspond to the N-terminal and C-terminal proteolytic fragments of the mature protein. Bioactive fragments of hedgehog polypeptides are described in great detail in PCT publications WO 95/18856 and WO 96/17924.

With respect to bioctive fragments of hedgehog polypeptide, preferred hedgehog therapeutics include at least 50 (contiguous) amino acid residues of a hedgehog polypeptide, more preferably at least 100 (contiguous), and even more preferably at least 150 (contiguous) residues.

Another preferred hedgehog polypeptide which can be included in the hedgehog therapeutic is an N-terminal fragment of the mature protein having a molecular weight of approximately 19 kDa.

Preferred human hedgehog proteins include N-terminal fragments corresponding approximately to residues 24-197 of SEQ ID No. 15, 28-202 of SEQ ID No. 16, and 23-198 of SEQ ID No. 17. By “corresponding approximately” it is meant that the sequence of interest is at most 20 amino acid residues different in length to the reference sequence, though more preferably at most 5, 10 or 15 amino acid different in length.

As described above for recombinant polypeptides, isolated hedgehog polypeptides can include all or a portion of the amino acid sequences represented in SEQ ID No: 8, SEQ ID No: 9, SEQ ID No: 10, SEQ ID No: 11, SEQ ID No: 12, SEQ ID No: 13 or SEQ ID No: 14, or a homologous sequence thereto. Preferred fragments of the subject hedgehog proteins correspond to the N-terminal and C-terminal proteolytic fragments of the mature protein. Bioactive fragments of hedgehog polypeptides are described in great detail in PCT publications WO 95/18856 and WO 96/17924.

Still other preferred hedgehog polypeptides includes an amino acid sequence represented by the formula A-B wherein: (i) A represents all or the portion of the amino acid sequence designated by residues 1-168 of SEQ ID No: 21; and B represents at least one amino acid residue of the amino acid sequence designated by residues 169-221 of SEQ ID No: 21; (ii) A represents all or the portion of the amino acid sequence designated by residues 24-193 of SEQ ID No: 15; and B represents at least one amino acid residue of the amino acid sequence designated by residues 194-250 of SEQ ID No: 15; (iii) A represents all or the portion of the amino acid sequence designated by residues 25-193 of SEQ ID No: 13; and B represents at least one amino acid residue of the amino acid sequence designated by residues 194-250 of SEQ ID No: 13; (iv) A represents all or the portion of the amino acid sequence designated by residues 23-193 of SEQ ID No: 11; and B represents at least one amino acid residue of the amino acid sequence designated by residues 194-250 of SEQ ID No: 11; (v) A represents all or the portion of the amino acid sequence designated by residues 28-197 of SEQ ID No: 12; and B represents at least one amino acid residue of the amino acid sequence designated by residues 198-250 of SEQ ID No: 12; (vi) A represents all or the portion of the amino acid sequence designated by residues 29-197 of SEQ ID No: 16; and B represents at least one amino acid residue of the amino acid sequence designated by residues 198-250 of SEQ ID No: 16; or (vii) A represents all or the portion of the amino acid sequence designated by residues 23-193 of SEQ ID No. 17, and B represents at least one amino acid residue of the amino acid sequence designated by residues 194-250 of SEQ ID No. 17. In certain preferred embodiments, A and B together represent a contiguous polypeptide sequence designated sequence, A represents at least 25, 50, 75, 100, 125 or 150 (contiguous) amino acids of the designated sequence, and B represents at least 5, 10, or 20 (contiguous) amino acid residues of the amino acid sequence designated by corresponding entry in the sequence listing, and A and B together preferably represent a contiguous sequence corresponding to the sequence listing entry. Similar fragments from other hedgehog also contemplated, e.g., fragments which correspond to the preferred fragments from the sequence listing entries which are enumerated above. In preferred embodiments, the hedgehog polypeptide includes a C-terminal glycine (or other appropriate residue) which is derivatized with a cholesterol.

Isolated peptidyl portions of hedgehog proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a hedgehog polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) hedgehog protein. For example, Román et al. (1994) Eur J Biochem 222:65-73 describe the use of competitive-binding assays using short, overlapping synthetic peptides from larger proteins to identify binding domains.

The recombinant hedgehog polypeptides of the present invention also include homologs of the authentic hedgehog proteins, such as versions of those protein which are resistant to proteolytic cleavage, as for example, due to mutations which alter potential cleavage sequences or which inactivate an enzymatic activity associated with the protein. Hedgehog homologs of the present invention also include proteins which have been post-translationally modified in a manner different than the authentic protein. Exemplary derivatives of hedgehog proteins include polypeptides which lack N-glycosylation sites (e.g. to produce an unglycosylated protein), which lack sites for cholesterolization, and/or which lack N-terminal and/or C-terminal sequences.

Modification of the structure of the subject hedgehog polypeptides can also be for such purposes as enhancing therapeutic or prophylactic efficacy, or stability (e.g., ex vivo shelf life and resistance to proteolytic degradation in vivo). Such modified peptides, when designed to retain at least one activity of the naturally-occurring form of the protein, are considered functional equivalents of the hedgehog polypeptides described in more detail herein. Such modified peptides can be produced, for instance, by amino acid substitution, deletion, or addition.

It is well known in the art that one could reasonably expect that certain isolated replacements of amino acids, e.g., replacement of an amino acid residue with another related amino acid (i.e. isosteric and/or isoelectric mutations), can be carried out without major effect on the biological activity of the resulting molecule. Conservative replacements are those that take place within a family of amino acids that are related in their side chains. Genetically encoded amino acids are can be divided into four families: (1) acidic=aspartate, glutamate; (2) basic lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W H Freeman and Co.: 1981). Whether a change in the amino acid sequence of a peptide results in a functional hedgehog homolog (e.g. functional in the sense that it acts to mimic or antagonize the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

It is specifically contemplated that the methods of the present invention can be carried using homologs of naturally occurring hedgehog proteins. In one embodiment, the invention contemplates using hedgehog polypeptides generated by combinatorial mutagenesis. Such methods, as are known in the art, are convenient for generating both point and truncation mutants, and can be especially useful for identifying potential variant sequences (e.g. homologs) that are functional in binding to a receptor for hedgehog proteins. The purpose of screening such combinatorial libraries is to generate, for example, novel hedgehog homologs which can act as either agonists or antagonist. To illustrate, hedgehog homologs can be engineered by the present method to provide more efficient binding to a cognate receptor, such as patched, yet still retain at least a portion of an activity associated with hedgehog. Thus, combinatorially-derived homologs can be generated to have an increased potency relative to a naturally occurring form of the protein. Likewise, hedgehog homologs can be generated by the present combinatorial approach to act as antagonists, in that they are able to mimic, for example, binding to other extracellular matrix components (such as receptors), yet not induce any biological response, thereby inhibiting the action of authentic hedgehog or hedgehog agonists. Moreover, manipulation of certain domains of hedgehog by the present method can provide domains more suitable for use in fusion proteins, such as one that incorporates portions of other proteins which are derived from the extracellular matrix and/or which bind extracellular matrix components.

To further illustrate the state of the art of combinatorial mutagenesis, it is noted that the review article of Gallop et al. (1994) J Med Chem 37:1233 describes the general state of the art of combinatorial libraries as of the earlier 1990's . In particular, Gallop et al state at page 1239 “[s]creening the analog libraries aids in determining the minimum size of the active sequence and in identifying those residues critical for binding and intolerant of substitution”. In addition, the Ladner et al. PCT publication WO90/02809, the Goeddel et al. U.S. Pat. No. 5,223,408, and the Markland et al. PCT publication WO92/15679 illustrate specific techniques which one skilled in the art could utilize to generate libraries of hedgehog variants which can be rapidly screened to identify variants/fragments which retained a particular activity of the hedgehog polypeptides. These techniques are exemplary of the art and demonstrate that large libraries of related variants/truncants can be generated and assayed to isolate particular variants without undue experimentation. Gustin et al. (1993) Virology 193:653, and Bass et al. (1990) Proteins: Structure, Function and Genetics 8:309-314 also describe other exemplary techniques from the art which can be adapted as means for generating mutagenic variants of hedgehog polypeptides.

Indeed, it is plain from the combinatorial mutagenesis art that large scale mutagenesis of hedgehog proteins, without any preconceived ideas of which residues were critical to the biological function, and generate wide arrays of variants having equivalent biological activity. Indeed, it is the ability of combinatorial techniques to screen billions of different variants by high throughout analysis that removes any requirement of a priori understanding or knowledge of critical residues.

To illustrate, the amino acid sequences for a population of hedgehog homologs or other related proteins are aligned, preferably to promote the highest homology possible. Such a population of variants can include, for example, hedgehog homologs from one or more species. Amino acids which appear at each position of the aligned sequences are selected to create a degenerate set of combinatorial sequences. In a preferred embodiment, the variegated library of hedgehog variants is generated by combinatorial mutagenesis at the nucleic acid level, and is encoded by a variegated gene library. For instance, a mixture of synthetic oligonucleotides can be enzymatically ligated into gene sequences such that the degenerate set of potential hedgehog sequences are expressible as individual polypeptides, or alternatively, as a set of larger fusion proteins (e.g. for phage display) containing the set of hedgehog sequences therein.

As illustrated in PCT publication WO 95/18856, to analyze the sequences of a population of variants, the amino acid sequences of interest can be aligned relative to sequence homology. The presence or absence of amino acids from an aligned sequence of a particular variant is relative to a chosen consensus length of a reference sequence, which can be real or artificial.

In an illustrative embodiment, alignment of exons 1, 2 and a portion of exon 3 encoded sequences (e.g. the N-terminal approximately 221 residues of the mature protein) of each of the Shh clones produces a degenerate set of Shh polypeptides represented by the general formula:

(SEQ ID No:21)

C-G-P-G-R-G-X(1)-G-X(2)-R-R-H-P-K-K-L-T-P-L-A-Y-K-
Q-F-I-P-N-V-A-E-K-T-L-G-A-S-G-R-Y-E-G-K-I-X(3)-R-
N-S-E-R-F-K-E-L-T-P-N-Y-N-P-D-I-I-F-K-D-E-E-N-T-G-
A-D-R-L-M-T-Q-R-C-K-D-K-L-N-X(4)-L-A-I-S-V-M-N-X
(5)-W-P-G-V-X(6)-L-R-V-T-E-G-W-D-E-D-G-H-H-X(7)-E-
E-S-L-H-Y-E-G-R-A-V-D-I-T-T-S-D-R-D-X(8)-S-K-Y-G-X
(9)-L-X(10)-R-L-A-V-E-A-G-F-D-W-V-Y-Y-E-S-K-A-H-I-
H-C-S-V-K-A-E-N-S-V-A-A-K-S-G-G-C-F-P-G-S-A-X(11)-
V-X(12)-L-X(13)-X(14)-G-G-X(15)-K-X-(16)-V-K-D-L-X
(17)-P-G-D-X(18)-V-L-A-A-D-X(19)-X(20)-G-X(21)-L-X
(22)-X(23)-S-D-F-X(24)-X(25)-F-X(26)-D-R

wherein each of the degenerate positions “X” can be an amino acid which occurs in that position in one of the human, mouse, chicken or zebrafish Shh clones, or, to expand the library, each X can also be selected from amongst amino acid residue which would be conservative substitutions for the amino acids which appear naturally in each of those positions. For instance, Xaa(1) represents Gly, Ala, Val, Leu, Ile, Phe, Tyr or Trp; Xaa(2) represents Arg, His or Lys; Xaa(3) represents Gly, Ala, Val, Leu, Ile, Ser or Thr; Xaa(4) represents Gly, Ala, Val, Leu, Ile, Ser or Thr; Xaa(5) represents Lys, Arg, His, Asn or Gln; Xaa(6) represents Lys, Arg or His; Xaa(7) represents Ser, Thr, Tyr, Trp or Phe; Xaa(8) represents Lys, Arg or His; Xaa(9) represents Met, Cys, Ser or Thr; Xaa(10) represents Gly, Ala, Val, Leu, Ile, Ser or Thr; Xaa(11) represents Leu, Val, Met, Thr or Ser; Xaa(12) represents His, Phe, Tyr, Ser, Thr, Met or Cys; Xaa(13) represents Gln, Asn, Glu, or Asp; Xaa(14) represents His, Phe, Tyr, Thr, Gln, Asn, Glu or Asp; Xaa(15) represents Gln, Asn, Glu, Asp, Thr, Ser, Met or Cys; Xaa(16) represents Ala, Gly, Cys, Leu, Val or Met; Xaa(17) represents Arg, Lys, Met, Ile, Asn, Asp, Glu, Gln, Ser, Thr or Cys; Xaa(18) represents Arg, Lys, Met or Ile; Xaa(19) represents Ala, Gly, Cys, Asp, Glu, Gln, Asn, Ser, Thr or Met; Xaa(20) represents Ala, Gly, Cys, Asp, Asn, Glu or Gln; Xaa(21) represents Arg, Lys, Met, Ile, Asn, Asp, Glu or Gln; Xaa(22) represent Leu, Val, Met or Ile; Xaa(23) represents Phe, Tyr, Thr, His or Trp; Xaa(24) represents Ile, Val, Leu or Met; Xaa(25) represents Met, Cys, Ile, Leu, Val, Thr or Ser; Xaa(26) represents Leu, Val, Met, Thr or Ser. In an even more expansive library, each X can be selected from any amino acid.

In similar fashion, alignment of each of the human, mouse, chicken and zebrafish hedgehog clones, can provide a degenerate polypeptide sequence represented by the general formula:

(SEQ ID No:22)

C-G-P-G-R-G-X(1)-X(2)-X(3)-R-R-X(4)-X(5)-X(6)-P-K-
X(7)-L-X(8)-P-L-X(9)-Y-K-Q-F-X(10)-P-X(11)-X(12)-X
(13)-E-X(14)-T-L-G-A-S-G-X(15)-X(16)-E-G-X(17)-X
(18)-X(19)-R-X(20)-S-E-R-F-X(21)-X(22)-L-T-P-N-Y-
N-P-D-I-I-F-K-D-E-E-N-X(23)-G-A-D-R-L-M-T-X(24)-R-
C-K-X(25)-X(26)-X(27)-N-X(28)-L-A-I-S-V-M-N-X(29)-
W-P-G-V-X(30)-L-R-V-T-E-G-X(31)-D-E-D-G-H-H-X(32)-
X(33)-X(34)-S-L-H-Y-E-G-R-A-X(35)-D-I-T-T-S-D-R-D-
X(36)-X(37)-K-Y-G-X(3 8)-L-X(39)-R-L-A-V-E-A-G-F-
D-W-V-Y-Y-E-S-X(40)-X(41)-H-X(42)-H-X(43)-S-V-K-X
(44)-X(45)

wherein, as above, each of the degenerate positions “X” can be an amino acid which occurs in a corresponding position in one of the wild-type clones, and may also include amino acid residue which would be conservative substitutions, or each X can be any amino acid residue. In an exemplary embodiment, Xaa(1) represents Gly, Ala, Val, Leu, Ile, Pro, Phe or Tyr; Xaa(2) represents Gly, Ala, Val, Leu or Ile; Xaa(3) represents Gly, Ala, Val, Leu, Ile, Lys, His or Arg; Xaa(4) represents Lys, Arg or His; Xaa(5) represents Phe, Trp, Tyr or an amino acid gap; Xaa(6) represents Gly, Ala, Val, Leu, Ile or an amino acid gap; Xaa(7) represents Asn, Gln, His, Arg or Lys; Xaa(8) represents Gly, Ala, Val, Leu, Ile, Ser or Thr; Xaa(9) represents Gly, Ala, Val, Leu, Ile, Ser or Thr; Xaa(10) represents Gly, Ala, Val, Leu, Ile, Ser or Thr; Xaa(11) represents Ser, Thr, Gln or Asn; Xaa(12) represents Met, Cys, Gly, Ala, Val, Leu, Ile, Ser or Thr; Xaa(13) represents Gly, Ala, Val, Leu, Ile or Pro; Xaa(14) represents Arg, His or Lys; Xaa(15) represents Gly, Ala, Val, Leu, Ile, Pro, Arg, His or Lys; Xaa(16) represents Gly, Ala, Val, Leu, Ile, Phe or Tyr; Xaa(17) represents Arg, His or Lys; Xaa(18) represents Gly, Ala, Val, Leu, Ile, Ser or Thr; Xaa(19) represents Thr or Ser; Xaa(20) represents Gly, Ala, Val, Leu, Ile, Asn or Gln; Xaa(21) represents Arg, His or Lys; Xaa(22) represents Asp or Glu; Xaa(23) represents Ser or Thr; Xaa(24) represents Glu, Asp, Gln or Asn; Xaa(25) represents Glu or Asp; Xaa(26) represents Arg, His or Lys; Xaa(27) represents Gly, Ala, Val, Leu or Ile; Xaa(28) represents Gly, Ala, Val, Leu, Ile, Thr or Ser; Xaa(29) represents Met, Cys, Gln, Asn, Arg, Lys or His; Xaa(30) represents Arg, His or Lys; Xaa(31) represents Trp, Phe, Tyr, Arg, His or Lys; Xaa(32) represents Gly, Ala, Val, Leu, Ile, Ser, Thr, Tyr or Phe; Xaa(33) represents Gln, Asn, Asp or Glu; Xaa(34) represents Asp or Glu; Xaa(35) represents Gly, Ala, Val, Leu, or Ile; Xaa(36) represents Arg, His or Lys; Xaa(37) represents Asn, Gln, Thr or Ser; Xaa(38) represents Gly, Ala, Val, Leu, Ile, Ser, Thr, Met or Cys; Xaa(39) represents Gly, Ala, Val, Leu, Ile, Thr or Ser; Xaa(40) represents Arg, His or Lys; Xaa(41) represents Asn, Gln, Gly, Ala, Val, Leu or Ile; Xaa(42) represents Gly, Ala, Val, Leu or Ile; Xaa(43) represents Gly, Ala, Val, Leu, Ile, Ser, Thr or Cys; Xaa(44) represents Gly, Ala, Val, Leu, Ile, Thr or Ser; and Xaa(45) represents Asp or Glu.

There are many ways by which the library of potential hedgehog homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential hedgehog sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis of hedgehog homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate hedgehog sequences created by combinatorial mutagenesis techniques.

In one embodiment, the combinatorial library is designed to be secreted (e.g. the polypeptides of the library all include a signal sequence but no transmembrane or cytoplasmic domains), and is used to transfect a eukaryotic cell that can be co-cultured with peripehral nerve cells. A functional hedgehog protein secreted by the cells expressing the combinatorial library will diffuse to neighboring peripheral nerve cells and induce a particular biological response, such as proliferation or differentiation. The pattern of detection of such a change in phenotype will resemble a gradient function, and will allow the isolation (generally after several repetitive rounds of selection) of cells producing hedgehog homologs active as neurotrophic agents. Likewise, hedgehog antagonists can be selected in similar fashion by the ability of the cell producing a functional antagonist to protect neighboring cells (e.g., to inhibit proliferation) from the effect of wild-type hedgehog added to the culture media.

To illustrate, target peripheral nerve cells are cultured in 24-well microtitre plates. Other eukaryotic cells are transfected with the combinatorial hedgehog gene library and cultured in cell culture inserts (e.g. Collaborative Biomedical Products, Catalog #40446) that are able to fit into the wells of the microtitre plate. The cell culture inserts are placed in the wells such that recombinant hedgehog homologs secreted by the cells in the insert can diffuse through the porous bottom of the insert and contact the target cells in the microtitre plate wells. After a period of time sufficient for functional forms of a hedgehog protein to produce a measurable response in the target cells, such as growth state, the inserts are removed and the effect of the variant hedgehog proteins on the target cells determined. Cells from the inserts corresponding to wells which score positive for activity can be split and re-cultured on several inserts, the process being repeated until the active clones are identified.

In yet another screening assay, the candidate hedgehog gene products are displayed on the surface of a cell or viral particle, and the ability of particular cells or viral particles to associate with a hedgehog-binding moiety (such as the patched protein or other hedgehog receptor) via this gene product is detected in a “panning assay”. Such panning steps can be carried out on cells cultured from embryos. For instance, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, fluorescently labeled molecules which bind hedgehog can be used to score for potentially functional hedgehog homologs. Cells can be visually inspected and separated under a fluorescence microscope, or, where the morphology of the cell permits, separated by a fluorescence-activated cell sorter.

In an alternate embodiment, the gene library is expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and f1 are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461).

In an illustrative embodiment, the recombinant phage antibody system (RPAS, Pharamacia Catalog number 27-9400-01) can be easily modified for use in expressing and screening hedgehog combinatorial libraries. For instance, the pCANTAB 5 phagemid of the RPAS kit contains the gene which encodes the phage gill coat protein. The hedgehog combinatorial gene library can be cloned into the phagemid adjacent to the gIII signal sequence such that it will be expressed as a gIII fusion protein. After ligation, the phagemid is used to transform competent E. coli TG1 cells. Transformed cells are subsequently infected with M13KO7 helper phage to rescue the phagemid and its candidate hedgehog gene insert. The resulting recombinant phage contain phagemid DNA encoding a specific candidate hedgehog, and display one or more copies of the corresponding fusion coat protein. The phage-displayed candidate hedgehog proteins which are capable of binding an hedgehog receptor are selected or enriched by panning. For instance, the phage library can be applied to cells which express the patched protein and unbound phage washed away from the cells. The bound phage is then isolated, and if the recombinant phage express at least one copy of the wild type gIII coat protein, they will retain their ability to infect E. coli. Thus, successive rounds of reinfection of E. coli, and panning will greatly enrich for hedgehog homologs, which can then be screened for further biological activities in order to differentiate agonists and antagonists.

Combinatorial mutagenesis has a potential to generate very large libraries of mutant proteins, e.g., in the order of 10²⁶ molecules. Combinatorial libraries of this size may be technically challenging to screen even with high throughput screening assays such as phage display. To overcome this problem, a new technique has been developed recently, recursive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the hedgehog protein to generate mimetics, e.g. peptide or non-peptide agents, which are able to disrupt binding of a hedgehog polypeptide of the present invention with an hedgehog receptor. Thus, such mutagenic techniques as described above are also useful to map the determinants of the hedgehog proteins which participate in protein-protein interactions involved in, for example, binding of the subject hedgehog polypeptide to other extracellular matrix components. To illustrate, the critical residues of a subject hedgehog polypeptide which are involved in molecular recognition of an hedgehog receptor such as patched can be determined and used to generate hedgehog-derived peptidomimetics which competitively inhibit binding of the authentic hedgehog protein with that moiety. By employing, for example, scanning mutagenesis to map the amino acid residues of each of the subject hedgehog proteins which are involved in binding other extracellular proteins, peptidomimetic compounds can be generated which mimic those residues of the hedgehog protein which facilitate the interaction. Such mimetics may then be used to interfere with the normal function of a hedgehog protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun126:419; and Dann et al. (1986) Biochem Biophys Res Commun 134:71).

Recombinantly produced forms of the hedgehog proteins can be produced using, e.g, expression vectors containing a nucleic acid encoding a hedgehog polypeptide, operably linked to at least one transcriptional regulatory sequence. Operably linked is intended to mean that the nucleotide sequence is linked to a regulatory sequence in a manner which allows expression of the nucleotide sequence. Regulatory sequences are art-recognized and are selected to direct expression of a hedgehog polypeptide. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences, sequences that control the expression of a DNA sequence when operatively linked to it, may be used in these vectors to express DNA sequences encoding hedgehog polypeptide. Such useful expression control sequences, include, for example, a viral LTR, such as the LTR of the Moloney murine leukemia virus, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

In addition to providing a ready source of hedgehog polypeptides for purification, the gene constructs of the present invention can also be used as a part of a gene therapy protocol to deliver nucleic acids encoding either an agonistic or antagonistic form of a hedgehog polypeptide. Thus, another aspect of the invention features expression vectors for in vivo transfection of a hedgehog polypeptide in particular cell types so as cause ectopic expression of a hedgehog polypeptide in an periperal neurons or other cells associated therewith.

Formulations of such expression constructs may be administered in any biologically effective carrier, e.g. any formulation or composition capable of effectively delivering the recombinant gene to cells in vivo. Approaches include insertion of the hedgehog coding sequence in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Furthermore, it will be recognized that the particular gene construct provided for in vivo transduction of hedgehog expression are also useful for in vitro transduction of cells, such as for use in the ex vivo tissue culture systems described below.

A preferred approach for in vivo introduction of nucleic acid into a cell is by use of a viral vector containing nucleic acid, e.g. a cDNA, encoding the particular form of the hedgehog polypeptide desired. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a hedgehog polypeptide and renders the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include Crip, Cre, 2 and Am. Retroviruses have been used to introduce a variety of genes into many different cell types, including neuronal cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface receptor ligands to the viral env proteins (Neda et al. (1991) J Biol Chem 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the hedgehog gene of the retroviral vector.

Another viral gene delivery system useful in the present method utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they can be used to infect a wide variety of cell types, including peripheral nerve cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the inserted hedgehog gene can be under control of, for example, the E1A promoter, the major late promoter (MLP) and associated leader sequences, the E3 promoter, or exogenously added promoter sequences.

In addition to viral transfer methods, such as those illustrated above, non-viral methods can also be employed to cause expression of a hedgehog polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the hedgehog polypeptide gene by the targeted cell. Exemplary gene delivery systems of this type include liposomal derived systems, poly-lysine conjugates, and artificial viral envelopes.

In clinical settings, the gene delivery systems for the therapeutic hedgehog gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057). A hedgehog expression construct can be delivered in a gene therapy construct to dermal cells by, e.g., electroporation using techniques described, for example, by Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

In yet another embodiment, the hedgehog or ptc therapeutic can be a “gene activation” construct which, by homologous recombination with a genomic DNA, alters the transcriptional regulatory sequences of an endogenous gene. For instance, the gene activation construct can replace the endogenous promoter of a hedgehog gene with a heterologous promoter, e.g., one which causes consitutive expression of the hedgehog gene or which causes inducible expression of the gene under conditions different from the normal expression pattern of the gene. Other genes in the patched signaling pathway can be similarly targeted. A vareity of different formats for the gene activation constructs are available. See, for example, the Transkaryotic Therapies, Inc PCT publications WO93/09222, WO95/31560, WO96/29411, WO95/31560 and WO94/12650.

In preferred embodiments, the nucleotide sequence used as the gene activation construct can be comprised of (1) DNA from some portion of the endogenous hedgehog gene (exon sequence, intron sequence, promoter sequences, etc.) which direct recombination and (2) heterologous transcriptional regulatory sequence(s) which is to be operably linked to the coding sequence for the genomic hedgehog gene upon recombination of the gene activation construct. For use in generating cultures of hedgehog producing cells, the construct may further include a reporter gene to detect the presence of the knockout construct in the cell.

The gene activation construct is inserted into a cell, and integrates with the genomic DNA of the cell in such a position so as to provide the heterologous regulatory sequences in operative association with the native hedgehog gene. Such insertion occurs by homologous recombination, i.e., recombination regions of the activation construct that are homologous to the endogenous hedgehog gene sequence hybridize to the genomic DNA and recombine with the genomic sequences so that the construct is incorporated into the corresponding position of the genomic DNA.

The terms “recombination region” or “targeting sequence” refer to a segment (i.e., a portion) of a gene activation construct having a sequence that is substantially identical to or substantially complementary to a genomic gene sequence, e.g., including 5′ flanking sequences of the genomic gene, and can facilitate homologous recombination between the genomic sequence and the targeting transgene construct.

As used herein, the term “replacement region” refers to a portion of a activation construct which becomes integrated into an endogenous chromosomal location following homologous recombination between a recombination region and a genomic sequence.

The heterologous regulatory sequences, e.g., which are provided in the replacement region, can include one or more of a variety elements, including: promoters (such as constitutive or inducible promoters), enhancers, negative regualtory elements, locus control regions, transcription factor binding sites, or combinations thereof.

Promoters/enhancers which may be used to control the expression of the targeted gene in vivo include, but are not limited to, the cytomegalovirus (CMV) promoter/enhancer (Karasuyama et al., 1989, J. Exp. Med., 169:13), the human β-actin promoter (Gunning et al. (1987) PNAS 84:4831-4835), the glucocorticoid-inducible promoter present in the mouse mammary tumor virus long terminal repeat (MMTV LTR) (Klessig et al. (1984) Mol. Cell Biol. 4:1354-1362), the long terminal repeat sequences of Moloney murine leukemia virus (MuLV LTR) (Weiss et al. (1985) RNA Tumor Viruses, Cold Spring Harbor Laboratory, Cold Spring Harbor, N.Y.), the SV40 early or late region promoter (Bernoist et al. (1981) Nature 290:304-310; Templeton et al. (1984) Mol. Cell Biol., 4:817; and Sprague et al. (1983) J. Virol., 45:773), the promoter contained in the 3′ long terminal repeat of Rous sarcoma virus (RSV) (Yamamoto et al., 1980, Cell, 22:787-797), the herpes simplex virus (HSV) thymidine kinase promoter/enhancer (Wagner et al. (1981) PNAS 82:3567-71), and the herpes simplex virus LAT promoter (Wolfe et al. (1992) Nature Genetics, 1:379-384).

In an exemplary embodiment, portions of the 5′ flanking region of the human Shh gene are amplified using primers which add restriction sites, to generate the following fragments

5′-gcgcgcttcgaaGCGAGGCAGCCAGCGAGGGAGAGAGCGAGCGGGCG
AGCCGGAGC-GAGGAAatcgatgcgcgc (primer 1)
5′-gcgcgcagatctGGGAAAGCGCAAGAGAGAGCGCACACGCACACACC
CGCCGCGCG-CACTCGggatccgcgcgc (primer 2)

As illustrated, primer 1 includes a 5′ non-coding region of the human Shh gene and is flanked by an AsuII and ClaI restriction sites. Primer 2 includes a portion of the 5′ non-coding region immediately 3′ to that present in primer 1. The hedgehog gene sequence is flanked by XhoII and BamHI restriction sites. The purified amplimers are cut with each of the enzymes as appropriate.

The vector pCDNA1.1 (Invitrogen) includes a CMV promoter. The plasmid is cut with with AsuII, which cleaves just 3′ to the CMV promoter sequence. The AsuII/ClaI fragment of primer 1 is ligated to the AsuII cleavage site of the pcDNA vector. The ClaI/AsuII ligation destroys the AsuII site at the 3′ end of a properly inserted primer 1.

The vector is then cut with BamHI, and an XhoIIBamHI fragment of primer 2 is ligated to the BamHI cleavage site. As above, the BamHI/XhoII ligation destroys the BamHI site at the 5′ end of a properly inserted primer 2.

Individual colonies are selected, cut with AsuII and BamHI, and the size of the AsuII/BamHI fragment determined. Colonies in which both the primer 1 and primer 2 sequences are correctly inserted are further amplified, an cut with AsuII and BamHI to produce the gene activation construct

cgaagcgaggcagccagcgagggagagagcgagcgggcgagccggagcga
ggaaATCGAAGGTTCGAATCCTTCCCCCACCACCATCACTTTCAAAAGTC
CGAAAGAATCTGCTCCCTGCTTGTGTGTTGGAGGTCGCTGAGTAGTGCGC
GAGTAAAATTTAACCTACAACAAGGCAAGGCTTGACCGACAATTGCATGA
AGAATCTGCTTAGGGTTAGGCGTTTTGCGCTGCTTCGCGATGTACGGGCC
AGATATACGCGTTGACATTGATTATTGACTAGTTATTAATAGTAATCAAT
TACGGGGTCATTAGTTCATAGCCCATATATCGAGTTCCGCGTTACATAAC
TTACGGTAAATGGCCCGCCTGGCTGACCGCCCAACGACCCCCGCCCATTG
ACGTCAATAATGACGTATGTTCCCATAGTAACGCCAATAGGGACTTTCCA
TTGACGTCAATGGGTGGACTATTTACGGTAAACTGCCCACTTGGCACTAC
ATCAAGTGTATCATATGCCAAGTACGCCCCCTATTGACGTCAATGACGGT
AAATGGCCCGCCTGGCATTATGCCCAGTACATGACCTTATGGGACTTTCC
TACTTGGCAGTACATCTACGTATTAGTCATCGCTATTACCATGGTGATGC
GGTTTTGGCAGTACATCAATGGGCGTGGATAGCGGTTTGACTCACGGGGA
TTTCCAAGTCTCCACCCCATTGACGTCAATGGGAGTTTGTTTTGGCACCA
AAATCAACGGGACTTTCCAAAATGTCGTAACAACTCCGCCCCATTGACGC
AAATGGGCGGTAGGCGTGTACGGTGGGAGGTCTATATAAGCAGAGCTCTC
TGGCTAACTAGAGAACCCACTGCTTACTGGCTTATCGAAATTAATACGAC
TCACTATAGGGAGACCCAAGCTTGGTACCGAGCTCGGATCgatctgggaa
agcgcaagagagagcgcacacgcacacacccgccgcgcgcactcgg

In this construct, the flanking primer 1 and primer 2 sequences provide the recombination region which permits the insertion of the CMV promoter in front of the coding sequence for the human Shh gene. Other heterologous promoters (or other transcriptional regulatory sequences) can be inserted in a genomic hedgehog gene by a similar method.

In still other embodiments, the replacement region merely deletes a negative transcriptional control element of the native gene, e.g., to activate expression, or ablates a positive control element, e.g., to inhibit expression of the targeted gene.

IV. Exemplary Neurotrophic Factors

Neurotrophic factors assist in this process by helping to maintain appropriate connections, and promoting nerve cell survival. Nerve growth factor (NGF) and other growth factors act via cell surface receptors to prevent neuronal death in the contexts of neural development, neurodegenerative disease, ischemia, axotomy and excessive excitatory amino acids (reviewed in: Longo et al., Nerve Growth Factor actions in the PNS and CNS. Eds: S. E. Loughlin and J. H. Fallon, Academic Press; pp 209-256 (1993)). They also promote neural regeneration and enhance neuronal function. Neurotrophic factors also can affect the developmental fate of neuronal cells. Neurotrophic factors are secreted polypeptides that usually act locally, near the cells producing them. The major class of neurotrophic factors is the neurotrophins. Other secreted molecules with neurotrophic activity (that may be considered neurotrophic factors) include ciliary neurotrophic factor and fibroblast growth factor.

The neurotrophins are a family of proteins with powerful effects on the development of the nervous system. Nerve growth factor (NGF) is the founding member of the family. NGF is a secreted protein processed from a proprotein. Mature NGF exists as a complex of three subunits, α, β and γ, with a stoichiometry of α₂β₂γ₂. The β dimer is the active component. Each β monomer contains three disulfide bonds and the dimerization interface is a hydrophobic surface formed by three beta-strands.

Other members of the neurotrophin family include brain-derived neurotrophin (BDNF), neurotrophin-3 (NT-3), neurotrophin-4 (NT-4), neurotrophin-5 (NT-5), and neurotrophin-6 (NT-6). Each of the neurotrophins contains a set of six cysteins at analagous positions in the amino acid sequence, that can participate in the three disulfide bonds. In addition the processed portions of the neurotrophins share roughly 50% of the amino acids in common. The portions of the precursor sequences that are not represented in the mature neurotrophic factors differ more widely between family members.

In humans and other higher vertebrates, NGF, BDNF and NT-3 have all been identified, as well their respective genes. NT-4 was discovered in Xenopus laevis. NT-5 is believed to be the mammalian equivalent of NT-4. NT-4 and NT-5 are often referred to as NT-4/5. NT-6 has been identified in fish, but its presence in mammals has been determined. Neurotrophins often have cross-species effects. For example, NT-4 from Xenopus laevis can replace NT-5 in chicks.

Other neurotrophic factors include Glial Cell Line-Derived Neurotrophic Factor (GDNF), which demonstrates neurotrophic activity both, in vivo and in vitro, and is currently being investigated for the treatment of Parkinson's disease. Insulin and Insulin-like growth factors have been shown to stimulate growth of neurites in rat pheochromocytoma PC12 cells and in cultured sympathetic and sensory neurons) Recio-Pinto et al., J. Neurosci., 6, pp. 1211-1219 (1986). Insulin and Insulin-like growth factors also stimulate the regeneration of injured motor nerves in vivo and in vitro. Near et al., PNAS, pp. 89, 11716-11720 (1992); and Edbladh et al., Brain Res., 641, pp. 76-82 (1994). Similarly, fibroblast growth factor (FGF) stimulates neural proliferation, D. Gospodarowicz et al., Cell Differ., 19, p. 1 (1986) and growth, M. A. Walter et al., Lymphokine Cytokine Res., 12, p. 135 (1993). Some of the neurotrophic factors are described below in more detail:

Nerve Growth Factor

Nerve growth factor (NGF) is by far the most fully characterized of these neurotrophic molecules and has been shown, both in vitro and in vivo, to be essential for the survival of sympathetic and neural crest-derived sensory neurons during early development of both chick and rat (Levi-Montalcini and Angeletti, 1963, Develop. Biol. 7:653-659; Levi-Montalcini et al., 1968, Physiol. Rev. 48:524-569). Injections of purified NGF into the developing chick embryo have been found to cause increase in survival and hypertrophy of spinal sensory neurons and sympathetic neurons (Levi-Montalcini and Booker, 1960, Proc. Natl. Acad. Sci. U.S.A. 46:373-384; Hamburger et al., 1981, J. Neurosci. 1:60-71). Conversely, removal or sequestration of endogenous NGF by daily injection of anti-NGF antibodies into neonatal rats has been associated with virtual destruction of the sympathetic nervous system (Levi-Montalcini and Booker, 1960, Proc. Natl. Acad. Sci. 46:384-391; Levi-Montalcini and Angeletti, 1966, Pharmacol. Rev. 18:619-628). Exposure to NGF antibodies even earlier in development either by antibody injections in utero or by passive transplacental transfer of maternal antibodies has been shown to result in a substantial loss of neural crest-derived sensory neurons such as spinal and dorsomedial trigeminal sensory neurons (Goedert et al., 1984, Proc. Natl. Acad. Sci. U.S.A. 81:1580-1584; Gorin and Johnson, 1979, Proc. Natl. Acad. Sci. U.S.A. 76:5382-5386). In addition, it appears that NGF also influences the development and maintenance of specific populations of neurons in the central nervous system (Thoenen et al., 1987, Rev. Physiol. Biochem. Pharmacol. 109:145-178; Whittemore and Seiger, 1987, Brain Res. Rev. 12:439-464).

The abundance of NGF protein in mouse submaxillary gland allowed the primary sequence to be determined by relatively conventional protein chemistry (Angeletti and Bradshaw, 1971, Proc. Natl. Acad. Sci. 68:2417-2420). The NGF gene has now been cloned from many species, including mouse (Scott et al., 1983, Nature 302:538-540, human (Ullrich et al., 1983, Nature 303;821-825) (SEQ ID No: 23), cow and chick (Meier et al., 1986, EMBO J. 5:1489-1493), and rat (Whittemore et al., 1988, J. Neurosci. Res., 20:402-410) using essentially conventional molecular biology based on the availability of the protein sequence of mouse NGF to design suitable oligonucleotide probes. The availability of abundant NGF has also greatly facilitated studies on the NGF receptor, which have ultimately led to the molecular cloning of one component of the NGF receptor from human and rat (Johnson et al., 1986, Cell, 47:545-554; Radeke et al., 1987, Nature 325:593-597).

Brain-Derived Neurotrophic Factor

A neurotrophic activity capable of sustaining the survival of embryonic chick dorsal root ganglion neurons in vitro was identified in the “conditioned medium” in which rat C-6 glioma cells had been cultured (Barde et al., 1978, Nature 274:818). The activity was not neutralized by antibodies to mouse NGF, suggesting the presence of another neurotrophic factor in the conditioned medium. Similar activities that could not be blocked by NGF antibodies were subsequently reported in cultures of normal adult rat brain astroglial cells (Lindsay, 1979, Nature 282:80-82; Lindsay et al., 1982, Brain Res. 243:329-343) and in extracts of developing and adult rat brain (Barde et al., 1980, Proc. Natl. Acad. Sci. U.S.A. 77:1199-1203) and developing and mature chick spinal cord (Lindsay and Peters, 1984, Neurosci. 12:45-51). However, in no case was the active factor(s) isolated or identified, and it remains questionable as to whether the observed activities were due to the same or different factor(s).

Using pig brain as a starting material, Barde et al. (1982, EMBO J. 1:549-553) reported a factor, now termed brain-derived neurotrophic factor (BDNF), which appeared to promote the survival of dorsal root ganglion neurons from E10/E11 chick embryos. The neurotrophic activity was found to reside in a highly basic protein (isoelectric point, pI 10.1) which migrated during sodium dodecyl sulfate (SDS) gel electrophoresis as a single band of 12.3 kD molecular weight. It was noted that the highly basic nature and molecular size of BDNF were very similar to the NGF monomer.

The first demonstration of neuronal specificity of BDNF distinct from that of NGF was the demonstration in vitro that purified BDNF supports the survival of 40-50% of sensory neurons dissociated from the neural placode-derived nodose ganglion of the chick embryo at E6, E9 or E12 (Lindsay et al., 1985, J. Cell. Sci. Supp. 3:115-129). NGF was without apparent effect on these neurons either by itself or in conjunction with BDNF. It was later shown in explant culture studies that BDNF appeared to support survival and neurite outgrowth from other neural placode-derived sensory ganglia, including the petrosal, geniculate and ventrolateral trigeminal ganglia (Davies et al., 1986, J. Neurosci. 6:1897-1904), none of which have been found to be sensitive to NGF. In all of the above studies, neutralizing antibodies to NGF had no effect upon the observed activity of BDNF. In addition to its effects on cultured neurons from peripheral ganglia, BDNF was found to stimulate survival and neuronal differentiation of cells cultured from quail neural crest (Kalcheim and Gendreau, 1988, Develop. Brain Res. 41:79-86).

Two recent studies with BDNF (Kalcheim, et al., 1987, EMBO J. 1:2871-2873; Hofer and Barde, 1988, Nature 331:261-262) have, however, indicated a physiological role of BDNF in avian PNS development. If a mechanical barrier was placed in ovo at E3/E4 (embryonic day 3 or 4) between developing dorsal root ganglia (DRG) and their CNS target in the neural tube, many DRG neurons were observed to die (Kalcheim and Le Dourarin, 1986, Develop. Biol. 116:451-46). It was postulated that this neuronal death may have been due to deprivation from a CNS (neural tube) derived neurotrophic factor. It was subsequently observed that BDNF attached to a laminin-coated sialastic membrane could prevent this cell death (Kalcheim et al., 1987, EMBO J. 1:2871-2873). Injections of BDNF into developing quail eggs has been found to reduce naturally occurring cell death in the nodose ganglion, an effect not seen with NGF (Hofer and Barde, 1988, Nature 331:261-262). In addition to its effect on peripheral sensory neurons of both neural crest and neural placode origin, BDNF was found to support the survival of developing CNS neurons. Johnson et al. (1986, J. Neurosci. 6:3031-3938) presented data indicating that BDNF supports the survival of retinal ganglion cells cultured from E17 rat embryos. This extended previous studies which showed that conditioned media and brain extracts prepared from the target regions of retinal ganglion cells appeared to support the survival of these neurons (McCaffery et al., 1982, Ex. Brain Res. 48:37-386; Sarthy et al., 1983, J. Neurosci. 3:2532-2544; Turner et al., 1983, Dev. Brain Res. 6:77-83).

In addition to its effects on the survival of developing neurons in culture, BDNF has been shown to have effects on cultured adult peripheral and central nervous system neurons. BDNF, as well as NGF, has been shown to stimulate axonal regeneration from adult rat DRG neurons in culture (Lindsay, 1988, J. Neurosci. 1:2394-2405) although adult sensory neurons did not appear to require neurotrophic factors for maintenance in vitro over 3 or 4 weeks. Furthermore, in cultures of adult rat retina, BDNF was observed to promote both survival and axonal elongation from retinal ganglion cells (Thanos et al., 1989, Eur. J. Neurosci. 1:19-26). In addition BDNF has been shown to prolong the survival of cells in ventral mesencephalic cultures, as measured by the number of tyrosine hydroxylase positive cells visualized by immunocytochemistry. In addition, BDNF enhances the survival of cholinergic neurons in dissociated cell culture derived from the rat septal region.

Neurotrophin-3

The marked similarities between NGF and BDNF suggested that both may be members of a larger family of closely related neurotrophic molecules. When regions of homology were used to devise oligonucleotide primers for polymerase chain reaction to identify new members of the BDNF/NGF gene family, another member of the family, termed neurotrophin-3, was discovered and the NT-3 gene was cloned from mouse, rat, and human. The overall structure of mature mouse NT-3 protein, consisting of 119 amino acids with a computed pI of about 9.5, was found to resemble that established for NGF and BDNF; a putative signal sequence of 18 amino acids (showing 5 and 9 amino acid identities with BDNF and NGF, respectively) appears to be followed by a prosequence of 121 amino acids (as compared with a prosequence of 103 amino acids in mouse NGF and a prosequence of 112 amino acids in mouse BDNF). All 6 cysteine residues, known in NGF and BDNF to be involved in the formation of disulfide bridges (Leibrock et al., 1989, Nature 341:149-152;Angeletti, 1973, Biochem. 12:100-115) are amongst the conserved residues. Similarly, mature rat NT-3 appears to share 57% amino acid homology with rat NGF, and 58% amino acid homology with rat BDNF; 57 of the 120 residues (48%) appear to be shared by all three proteins. Again, the six cysteine residues of rat NGF and BDNF were found to be absolutely conserved in rat NT-3, and regions of greatest homology between the three proteins appear to cluster around these cysteine residues.

In addition to the homology between NT-3, NGF, and BDNF within a species, a high degree of conservation in nucleic acid sequence was observed between rat and human NT-3 within the region encoding the mature polypeptide (119 amino acids). The human and rat genes were found to be approximately 92% homologous in DNA sequence. However, none of the differences in nucleotide sequences between human and rat in this region lead to amino acid substitutions; the deduced amino acid sequences of mature rat and human (as well as mouse NT-3) appear absolutely identical, reminiscent of the high degree of conservation of BDNF, which shows complete identity in the amino acid sequence of the mature polypeptide among rat, mouse, human, and pig. By contrast, the amino acid sequences of mature human NGF and rodent NGF (mouse or rat) differ by approximately 10 percent.

Studies of the neurotrophic activity of NT-3 have indicated that NT-3 is capable of promoting survival and neurite outgrowth of dissociated dorsal root ganglion neurons in culture. Furthermore, NT-3 was observed to promote neurite outgrowth from both nodose ganglion and sympathetic ganglion explants, whereas BDNF promoted outgrowth from nodose ganglion but not sympathetic ganglion, and NGF promoted outgrowth from sympathetic ganglion but not nodose ganglion explants. Therefore, NT-3 appears to have a broader specificity of action than either BDNF or NGF.

NT-3 may be important in the development of the nervous system. When the relative abundance of NGF, BDNF, and NT-3 transcripts in the brains of newborn and adult mice were compared, the level of NT-3 in newborn brain was found to be higher than in adult brain. NT-3 RNA levels in the central nervous system were observed to be dramatically higher during fetal development and were found to subsequently decrease to adult levels.

Ciliary Neurotrophic Factor

Ciliary neurotrophic factors (CNTFs) are proteins that specifically promote the survival of embryonic chick ciliary ganglion neurons in vitro (Manthorpe et al., 1980, J. Neurochem. 34:69-75). The ciliary ganglion is anatomically located within the orbital cavity, lying between the lateral rectus and the sheath of the optic nerve; it receives parasympathetic nerve fibers from the oculomotor nerve which innervate the ciliary muscle and sphincter pupillae.

Ciliary ganglion neurons have been found to be among the neuronal populations which exhibit defined periods of cell death. In the chick ciliary ganglion, half of the neurons present at embryonic day 8 (E8) have been observed to die before E14 (Landmesser and Pilar, 1974, J. Physiol. 241: 737-749). During this same time period, ciliary ganglion neurons are forming connections with their target tissues, namely, the ciliary body and the choroid coat of the eye. Landmesser and Pilar (1974, J. Physiol. 241:751-736) observed that removal of an eye prior to the period of cell death results in the complete loss of ciliary ganglion neurons in the ipsilateral ganglion. Conversely, Narayanan and Narayanan (1978, J. Embryol. Ex. Morphol. 44:53-70) observed that, by implanting an additional eye primordium and thereby increasing the amount of available target tissue, ciliary ganglion neuronal cell death may be decreased. These results are consistent with the existence of a neurotrophic factor which acts upon ciliary ganglion neurons.

In culture, ciliary ganglion (CG) neurons have been found to require a factor or factors for survival. Ciliary neurotrophic factor(s) (CNTF) activity has been identified in chick muscle cell conditioned media (Helfand et al., 1976, Dev. Biol. 50:541-547; Helfand et al., 1978, Exp. Cell Es. 113:39-45; Bennett and Nurcombe, 1979, Brain Res. 173:543-548; Nishi and Berg, 1979, Nature 277:232-234; Varon et al., 1979, Brain Res. 173:29-45), in muscle extracts (McLennan and Hendry, 1978, Neurosci. Lett. 10:269-273; Bonahandy et al., 1980, Neurosci. Lett. 18:197-201), in chick embryo extract (Varon et al., n1979, Brain Res. 173:29-45; Tuttle et al., 1980, Brain Res. 183:161-180), and in medium conditioned by heart cells (for discussion, see also Adler et al., 1979, Science 204:1434-1436 and Barbin et al., 1984, J. Neurochem. 43:1468-1478).

CNTF has been purified from rat sciatic nerve and the amino acid sequence of various fragments determined by gas phase microsequencing; the resulting amino acid sequence was used to clone a rat CNTF gene using polymerase chain reaction-based cloning techniques. A rat CNTF probe were subsequently used in the cloning of the human CNTF gene. Comparison of the nucleic acid sequences of human and rat CNTF genes indicate that the human gene has a single intron at the same position as the rat CNTF gene. Within the intron, the human sequences appear to have diverged considerably from the rat, in marked contrast to substantial conservation of the coding region.

Based on nucleotide sequence, CNTF may be predicted to have a molecular weight of about 22.8 KD (calculated from an estimated size of about 200 amino acids), which is in agreement with that estimated for naturally occurring CNTF from polyacrylamide gel electrophoresis analysis (22.5 KD; Saadat et al., 1989, J. Cell Biol. 108:1807-1816). Thus, the amino acid sequence of CNTF shows the features of a cytosolic protein, i.e. no signal peptide, no consensus sequences for glycosylation and only one cysteine residue at position 17. No sequence homology was observed between CNTF and NGF, BDNF, or fibroblast growth factor (FGF) and purpurin, each of which are associated with survival activities similar to those of CNTF (Unsicker et al., 1987, Proc. Natl. Acad. Sci. U.S.A., 84:5459-5463; Schubert et al., 1986, J. Cell Biol. 102:2295-2301) A number of biological effects have been ascribed to CNTF. CNTF was originally described as an activity which supported the survival of neurons of the E8 chick ciliary ganglion, which is a component of the parasympathetic nervous system. Saadat et al., (1989, J. Cell Biol. 108:1807-1816) observed that their most highly purified preparation of rat sciatic nerve CNTF induced cholinergic differentiation of rat sympathetic neurons in culture. Also, Hoffman (1988, J. Neurochem. 51:109-113) found that CNTF activity derived from chick eye increased the level of choline-O-acetyltransferase activity in retinal monolayer cultures.

Hughes et al. (1988, Nature 335:70-73) studied a population of bipotential glial progenitor cells in the perinatal rat optic nerve and brain; this cell population is believed to give rise to, first, oligodendrocytes and then, second, to type 2 astrocytes. Studies have suggested that oligodendrocyte differentiation occurs from an oligodendrocyte-type 2-astrocyte (0-2A) progenitor cell in the absence of any particular growth factor, whereas type 2 astrocyte differentiation appears to require the presence of a specific inducing protein. Hughes et al. observed that the type 2 astrocyte inducing protein is similar or identical to CNTF (see also Anderson, 1989, Trends Neurosci. 12:83-85).

In addition, recombinant CNTF has been shown to promote the survival of mediodorsal spinal cord neurons in culture, and purified rat sciatic nerve CNTF was observed to prevent lesion-induced cell death of motorneurons in lesioned facial nerve of newborn rat.

In another embodiment, the invention also provides compounds displaying NGF agonist activity, examples include chimeric neurotrophic factors such as those disclose in U.S. Pat. No: 5,512,661, which provide, for example, the activity of two neurotrophic factors in a single molecule, or may serve as superagonists of an endogenous neurotrophic factor, thereby enabling an increased biological response at lower doses. Additionally the chimeric neurotrophic factors of the invention may be useful in targeting an active compound to cells responsive to neurotrophic factor. Furthermore, the design of chimeric neurotrophic factors which retain specific biological activity but which are directed to a subset of factor-responsive cells may enable the treatment of neurological disorders but avoid the complications of more widespread activity of a parental molecule(s). Similar neurotrophic agonist compounds include compounds comprising a multimer of a sequence of amino acid residues or biologically functional equivalents thereof, the sequence being substantially homologous to residues 29-38 of NGF, residues 43-47 of NGF or residues 92-97 of NGF. Preferably, the multimer is a cyclic dimer wherein the monomeric amino acid sequences are linked to each other by disulfide bridges. This invention also provides methods and compositions for the treatment of disorders involving NGF-responsive cells, by administering an effective amount of acomposition comprising a hedgehog therapeutic in combination with multimeric NGF agonists to an individual suffering from such disorder.

The compositions may include compounds displaying NGF agonist or partial agonist activity, wherein the compounds comprise a sequence of amino acid residues or biologically functional equivalents thereof, the sequence being substantially homologous to residues 29-38 of NGF, residues 43-47 of NGF or residues 92-97 of NGF, the sequence further comprising a penicillamine residue or a cysteine residue.

Also. Included are compositions comprising compounds displaying NGF agonist activity, wherein the compounds comprise a multimer of a sequence of amino acid residues or biologically functional equivalents thereof, the sequence being substantially homologous to residues 29-38 of NGF, residues 43-47 of NGF or residues 92-97 of NGF. Preferably, the multimer is a cyclic dimer wherein the monomeric amino acid sequences are linked to each other by disulfide bridges. The compounds of this invention promote neurite outgrowth and support neuronal survival.

Thus, it is expected that many variants of the known neurotrophins would also have significant effects on human neural development and function.

Neurotrophins and the Nervous System:

The neurotrophins have effects on both the peripheral and central nervous systems. In the peripheral nervous system, NGF is produced by the post-synaptic cell and promotes survival of the presynaptic neuron. Injections of anti-NGF antibody lead to NGF depletion and cause symptoms indicative of damage to neurons involved in nociceptive information. Gene knockout experiments suggest that, in mammals, 70-80% of the sensory neurons in the dorsal root ganglion (DRG) or the trigeminal ganglion depend on NGF during development. Other neurotrophins can partially replace NGF in several cell types, and vice versa. BDNF and NT-3 are both able to promote survival of various peripheral nerve cells, inlcuding placode-derived neurons, neural crest-derived neurons. NT-3 supports muscle-innervating sensory neurons in the DRG. NT-3 knockout mice suffer defects in proprioceptivce neurons, implying a function for NT-3 in these cell types. NT-3 may also have a mitogenic effect on newly migrated neural crest cells. Vestibular and auditory systems depend on NT-3 and/or BDNF. All of the neurotrophins may have an important role in the regeneration of damaged peripheral nerves, and promote neurite extension in vitro.

In the central nervous system, the neurotrophins have a major role in development and maintenance of neurons. In general, the effects of injury or age-related atrophy in vertebrates can be mitigated by administration of a member of the neurotrophin family. Lesion studies have shown that NGF can prevent death of axotomized septal neurons, and helps these neurons maintain cholinergic marker enzymes (such as choline acetyltransferase). NGF can also prevent atrophy of these neurons in aging rodents. BDNF, in particular, and the other neurotrophins may also play a role in development and survival of the cholinergic neurons of the septo-hippocampal system. Neural cell death in the isthmo-optic nucleus is decreased by treatment with BDNF or NT-3. Survival of retinal ganglion cells is promoted by treatment with NT-4/5 or BDNF. Motoneuron survival is increased in vitro by addition of BDNF, NT-4/5 or NT-3. Finally, each of the neurotrophins also appears to be capable of assisting in synaptic remodeling in either the developing or mature CNS.

V. Exemplary Applications of Method and Compositions

We have determined that hedgehog and ptc therapeutics lower the ED50 for neurotrophic factors to affect differentiation and/or survival of neuronal cells. Based in part on these findings, we have determined that this combination of agents is useful as protective agents in the treatment and prophylaxis for neurodegenerative disorders, including, without limitation, Alzheimer's disease, Parkinson's disease, Huntington's disease, amyotrophic lateral sclerosis and the like.

One aspect of the present invention relates to a method of enhancing differentiation and/or survival of neronal cells by contacting the cells with a trophic amount of a hedgehog or ptc thereapeutic conjointly with a neurotrophic factor. For instance, it is contemplated by the invention that, in light of the present finding of an apparently trophic effect of conjoint neurotrophic factor—hedgehog polypeptide treatment in the maintenance of differentiated neurons, the subject method could be used to maintain different neuronal tissue both in vitro and in vivo. Where the trophic agent includes a hedgehog polypeptide, it can be provided to a cell culture or animal as a purified protein or secreted by a recombinant cell, or cells or tissue explants which naturally produce one or more hedgehog polypeptides. For instance, neural tube explants from embryos, particularly floorplate tissue, can provide a source for Shh polypeptide, which source can be implanted in a patient or otherwise provided, as appropriate, for maintenance of differentiation. Where the trophic agent includes NGF, this invention provides compounds displaying NGF agonist or partial agonist activity, wherein the compounds comprise a sequence of amino acid residues or biologically functional equivalents thereof, the sequence being substantially homologous to residues 29-38 of NGF, residues 43-47 of NGF or residues 92-97 of NGF, the sequence further comprising a penicillamine residue or a cysteine residue. This invention provides compounds displaying NGF agonist activity, wherein the compounds comprise a multimer of a sequence of amino acid residues or biologically functional equivalents thereof, the sequence being substantially homologous to residues 29-38 of NGF, residues 43-47 of NGF or residues 92-97 of NGF. Preferably, the multimer is a cyclic dimer wherein the monomeric amino acid sequences are linked to each other by disulfide bridges. The compounds of this invention support neuronal survival and differentiation.

A. Cell Culture

The present method is applicable to cell culture techniques. In vitro neuronal culture systems have proved to be fundamental and indispensable tools for the study of neural development, as well as the identification of neurotrophic factors such as nerve growth factor (NGF), ciliary trophic factors (CNTF), and brain derived neurotrophic factor (BDNF). The present method provides a means for ensuring an adequately restrictive environment in order to maintain cholinergic, dopaminergic, GABAergic or other neuronal cells in differentiated states, and can be employed, for instance, in cell cultures designed to test the specific activities of other trophic factors.

In such embodiments of the subject method, a culture of differentiated cells including the neuronal cells can be contacted with a neurotrophic factor and a hedgehog or ptc therapeutic in order to maintain the integrity of a culture of terminally-differentiated neuronal cells by preventing loss of differentiation. The source of hedgehog or ptc therapeutic in the culture can be derived from, for example, a purified or semi-purified protein composition added directly to the cell culture media, or alternatively, supported and/or released from a polymeric device which supports the growth of various neuronal cells and which has been doped with the protein. The source of, for example, a trophic hedgehog polypeptide can also be a cell that is co-cultured with the neuronal cells. Alternatively, the source can be the neuronal cell itself which has been engineered to produce a recombinant hedgehog polypeptide. Such neuronal cultures can be used as convenient assay systems as well as sources of implantable cells for therapeutic treatments.

Cells can be obtained from embryonic, post-natal, juvenile or adult neural tissue from any animal. By any animal is meant any multicellular animal which contains nervous tissue. More particularly, is meant any fish, reptile, bird, amphibian or mammal and the like. The most preferable donors are mammals, especially humans and non-human primates, pigs, cows, and rodents.

Intracerebral neural grafting has emerged recently as an additional potential to CNS therapy. For example, one approach to repairing damaged brain tissues involves the transplantation of cells from fetal or neonatal animals into the adult brain (Dunnett et al. (1987) J Exp Biol 123:265-289; and Freund et al. (1985) J Neurosci 5:603-616). Fetal neurons from a variety of brain regions can be successfully incorporated into the adult brain, and such grafts can alleviate behavioral defects. For example, movement disorder induced by lesions of dopaminergic projections to the basal ganglia can be prevented by grafts of embryonic dopaminergic neurons. Complex cognitive functions that are impaired after lesions of the neocortex can also be partially restored by grafts of embryonic cortical cells. Transplantation of fetal brain cells, which contain precursors of the dopaminergic neurons, has been examined with success as a treatment for Parkinson's disease. In animal models and in patients with this disease, fetal brain cell transplantations have resulted in the reduction of motor abnormalities. Furthermore, it appears that the implanted fetal dopaminergic neurons form synapses with surrounding host neurons. However, in the art, the transplantation of fetal brain cells is limited due, for example, to the limited survival time of the implanted neuronal precursors and differentiated neurons arising therefrom. The subject invention provides a means for extending the usefulness of such transplants by enhancing the survival of dopaminergic and/or GABAergic cells in the transplant.

In the specific case of Parkinson's disease, intervention by increasing the activity of hedgehog in combination with a neurotrophic factor, by ectopic or endogenous means, can improve the in vivo survival of fetal and adult dopaminergic neurons, and thus can provide a more effective treatment of this disease. Cells to be transplanted for the treatment of a particular disease can be genetically modified in vitro so as to increase the expression of hedgehog in the transplant. In an exemplary embodiment of the invention, administration of an Shh polypeptide can be used in conjunction with surgical implantation of tissue in the treatment of Parkinson's disease.

In the case of a heterologous donor animal, the animal may be euthanized, and the brain and specific area of interest removed using a sterile procedure. Brain areas of particular interest include any area from which progenitor cells can be obtained which will provide dopaminergic or GABAergic cells upon differentiation. These regions include areas of the central nervous system (CNS) including the substantia nigra pars compacta which is found to be degenerated in Parkinson's Disease patients.

Human heterologous neural progenitor cells may be derived from fetal tissue obtained from elective abortion, or from a post-natal, juvenile or adult organ donor. Autologous neural tissue can be obtained by biopsy, or from patients undergoing neurosurgery in which neural tissue is removed, such as during epilepsy surgery.

Cells can be obtained from donor tissue by dissociation of individual cells from the connecting extracellular matrix of the tissue. Dissociation can be obtained using any known procedure, including treatment with enzymes such as trypsin, collagenase and the like, or by using physical methods of dissociation such as with a blunt instrument. Dissociation of fetal cells can be carried out in tissue culture medium, while a preferable medium for dissociation of juvenile and adult cells is artificial cerebral spinal fluid (aCSF). Regular aCSF contains 124 mM NaCl, 5 mM KCl, 1.3 mM MgCl₂, 2 mM CaCl₂, 26 mM NaHCO₃, and 10 mM D-glucose. Low Ca²⁺ aCSF contains the same ingredients except for MgCl₂ at a concentration of 3.2 mM and CaCl₂ at a concentration of 0.1 mM.

Dissociated cells can be placed into any known culture medium capable of supporting cell growth, including MEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. A particularly preferable medium for cells is a mixture of DMEM and F-12.

Conditions for culturing should be close to physiological conditions. The pH of the culture media should be close to physiological pH, preferably between pH 6-8, more preferably close to pH 7, even more particularly about pH 7.4. Cells should be cultured at a temperature close to physiological temperature, preferably between 30° C.-40° C., more preferably between 32° C.-38° C., and most preferably between 35° C.-37° C.

Cells can be grown in suspension or on a fixed substrate, but proliferation of the progenitors is preferably done in suspension to generate large numbers of cells by formation of “neurospheres” (see, for example, Reynolds et al. (1992) Science 255:1070-1709; and PCT Publications WO93/01275, WO94/09119, WO94/10292, and WO94/16718). In the case of propagating (or splitting) suspension cells, flasks are shaken well and the neurospheres allowed to settle on the bottom comer of the flask. The spheres are then transferred to a 50 ml centrifuge tube and centrifuged at low speed. The medium is aspirated, the cells resuspended in a small amount of medium with growth factor, and the cells mechanically dissociated and resuspended in separate aliquots of media.

Cell suspensions in culture medium are supplemented with any growth factor which allows for the proliferation of progenitor cells and seeded in any receptacle capable of sustaining cells, though as set out above, preferably in culture flasks or roller bottles. Cells typically proliferate within 3-4 days in a 37° C. incubator, and proliferation can be reinitiated at any time after that by dissociation of the cells and resuspension in fresh medium containing growth factors.

In the absence of substrate, cells lift off the floor of the flask and continue to proliferate in suspension forming a hollow sphere of undifferentiated cells. After approximately 3-10 days in vitro, the proliferating clusters (neurospheres) are fed every 2-7 days, and more particularly every 2-4 days by gentle centrifugation and resuspension in medium containing growth factor.

After 6-7 days in vitro, individual cells in the neurospheres can be separated by physical dissociation of the neurospheres with a blunt instrument, more particularly by triturating the neurospheres with a pipette. Single cells from the dissociated neurospheres are suspended in culture medium containing growth factors, and differentiation of the cells can be induced by plating (or resuspending) the cells in the presence of a factor capable of sustaining differentiation, e.g., such as a hedgehog or ptc therapeutic of the present invention.

Stem cells useful in the present invention are generally known. For example, several neural crest cells have been identified, some of which are multipotent and likely represent uncommitted neural crest cells. The hedgehog polypeptide can be used in combination with other neurotrophic factors which act to more particularly enhance a particular differentiation fate of the neuronal progenitor cell.

B. In vivo applications

In addition to the implantation of cells cultured in the presence of a combination of a neurotrophic factor and a functional hedgehog activity and other in vitro uses described above, yet another aspect of the present invention concerns the therapeutic application of a hedgehog or ptc therapeutic to enhance survival of neurons such as cholinergic, dopaminergic and GABAergic neurons. The ability of conjoint therapy to maintain neuronal differentiation indicates that certain of the hedgehog polypeptides can be reasonably expected to facilitate control of these neuronal cell-types in adult tissue with regard to maintenance, functional performance, aging and prevention of degeneration and premature death which result from loss of differentiation in certain pathological conditions.

In light of this understanding, the present invention specifically contemplates applications of the subject method to the treatment of degeneration of neuronal cells, including cell death or loss of functional performance, e.g., such as loss of dopaminergic cells, loss of GABAergic cells, and/or loss of neurons of the substantia nigra. In this regard, the subject method is useful in the treatment or prevention of such neurologic disorders including Parkinson's disease, domoic acid poisoning; spinal cord trauma; hypoglycemia; mechanical trauma to the nervous system; senile dementia; Korsakoff's disease; schizophrenia; AIDS dementia, multi-infarct dementia; mood disorders; depression; chemical toxicity and neuronal damage associated with uncontrolled seizures, such as epileptic seizures; and chronic neurologic disorders such as Huntington's disease, neuronal injury associated with HIV and AIDS, AIDS dementia, neurodegeneration associated with Down's syndrome, neuropathic pain syndrome, olivopontocerebral atrophy, amyotrophic lateral sclerosis, mitochondrial abnormalities, Alzheimer's disease, hepatic encephalopathy, Tourette's syndrome, schizophrenia, and drug addiction.

The subject therpeutics can also used to reduce neurotoxic injury associated with conditions of hypoxia, anoxia or ischemia which typically follows stroke, cerebrovascular accident, brain or spinal chord trauma, myocardial infarct, physical trauma, drownings, suffocation, perinatal asphyxia, or hypoglycemic events.

In preferred embodiments, the subject method is used to reduce the severity or prevent Alzheimer's disease.

In general, the therapeutic method of the present invention can be characterized as including a step of administering to an animal an amount of a ptc or hedgehog therapeutic in combination with a neurotrophic factor effective to enhance the survival of cholinergic, dopaminergic and/or GABAergic neuronal cells. The mode of administration and dosage regimens will vary depending on the severity of the degenerative disoder being treated, e.g., the dosage may be altered as between a prophylaxis and treatment.

The subject method may also find particular utility in treating or preventing the adverse neurological consequences of surgery. For example, certain cranial surgery can result in degeneration of neuronal populations for which the subject method can be applied.

In other embodiments, the subject method can be used to prevent or treat neurodegenerative conditions arising from the use of certain drugs, such as the compound MPTP (1-methyl-4-phenyl-1,2,3,6-tetrahydropyridine).

In still other embodiments, the subject method can be used in the prevention and/or treatment of hypoxia, e.g., as a neuroprotective agent. For instance, the subject method can be used prophylactically to lessen the neuronal cell death caused by altitude-induced hypoxia.

A method which is “neuroprotective”, in the case of dopaminergic and GABAergic cells, results in diminished loss of cells of those phenotype relative to that which would occur in the absence of treatment with a composition of the present invention, which composition comprises a hedgehog therapeutic in combination with a neurotrophic factor.

(i) Treatment of Alzheimer's Disease

Alzheimer's Disease is marked by widespread neurodegeneration in the brain including an enhanced loss of the cholinergic neurons that reside in the basal forebrain. This loss correlates to the severe cognitive deficits observed in Alzheimer's diseased patients. The induction of basal forebrain neurons during development has been shown to be dependent on exposure to the secreted inducing molecule sonic hedgehog (Shh) (Ericson et al., 1995). The loss of the basal forebrain cholinergic neurons contributes to the cognitive and spatial memory deficits in Alzheimer's diseased patients (Gilmor et al., 1999; Lehericy et al. 1993). According to the present invention, in vivo, treatment with the compositions comprising hedgehog in combination with other neurotrophic factors of the present invention, is expected to restore and modulate cholinergic function in Alzheimer's patients.

(ii) Treatment of Parkinson's Disease

It is now widely appreciated that the primary pathology underlying Parkinson's disease is degeneration of the dopaminergic projection from the substantia nigra to the striatum. This realisation has led to the widespread use of dopamine-replacing agents such as L-DOPA and apomorphine as symptomatic treatments for Parkinson's disease. Over the last three decades, such therapies have undoubtedly been successful in increasing the quality of life of patients suffering from Parkinson's disease, but, dopamine-replacement treatments do have limitations, especially following long-term treatment. Problems can include a wearing-off of anti-parkinsonian efficacy and the appearance of a range of dyskinesias characterised by chorea and dystonia. Ultimately, these side-effects can severely limit the usefulness of dopaminergic treatments.

Hedgehog therapeutics exerts trophic and survival-promoting actions on substantia nigra dopaminergic neurons. In vivo, treatment with the compositions comprising hedgehog in combination with other neurotrophic factors of the present invention, is expected to stimulate, for example, the dopaminergic phenotype of substantia nigra neurons and restore functional deficits induced by axotomy or dopaminergic neurotoxins. Therefore it may be used in the treatment of Parkinson's disease, a neurodegenerative disease characterized by the loss of dopaminergic neurons. Thus, in one embodiment, the subject method comprises administering to an animal afflicted with Parkinson's disease, or at risk of developing Parkonson's disease, an amount of the compositions of the present invention, effective for protecting or restoring the function of neurons affected by the Parkinson's condition, e.g., increasing the rate of survival of dopaminergic neurons in the animal. In preferred embodiments, the method includes administering to the animal an amount of the compositions of the present invention which would otherwise be effective at protecting the substantia nigra from MPTP-mediated toxicity when MPTP is administered at a dose of 0.5 mg/kg, more preferably at a dose of 2 mg/kg, 5 mg/kg, 10 mg/kg, 20 mg/kg or 50 mg/kg and, more preferably, at a dose of 100 mg/kg.

(iii) Treatment of Huntington's Disease

Huntington's disease involves the degeneration of intrastraital and cortical cholinergic neurons and GABAergic neurons. Treatment of patients suffering from such degenerative conditions can include the application of the compositions of the present invention, in order to control, for example, apoptotic events which give rise to loss of GABAergic neurons (e.g. to enhance survival of existing neurons.

(iv) Treatment of Amyotrophic Lateral Sclerosis

Recently it has been reported that in certain ALS patients and animal models a significant loss of midbrain dopaminergic neurons occurs in addition to the loss of spinal motor neurons. For instance, the literature describes degeneration of the substantia nigra in some patients with familial amyotrophic lateral sclerosis. Kostic et al. (1997) Ann Neurol 41:497-504. According to the subject invention, a trophic amount of compositions of the present invention can be administered to an animal suffering from, or at risk of developing, ALS.

(v) Treatment of Epilepsy

Epilepsy is a recurrent paroxysmal disorder of cerebral function characterized by sudden brief attacks of altered consciousness, motor activity, sensory phenomena or inappropriate behavior caused by abnormal excessive discharge of cerebral neurons. Convulsive seizures, the most common form of attacks, begin with loss of consciousness and motor control, and tonic or clonic jerking of all extremities but any recurrent seizure pattern may be termed epilepsy.

The term primary or idiopathic epilepsy denotes those cases where no cause for the seizures can be identified. Secondary or symptomatic epilepsy designates the disorder when it is associated with such factors as trauma, neoplasm, infection, developmental abnormalities, cerebrovascular disease, or various metabolic conditions. Epileptic seizures are classified as partial seizures (focal, local seizures) or generalized seizures (convulsive or nonconvulsive). Classes of partial seizures include simple partial seizures, complex partial seizures and partial seizures secondarily generalized. Classes of generalized seizures include absence seizures, atypical absence seizures, myoclonic seizures, clonic seizures, tonic seizures, tonic-clonic seizures (grand mal) and atonic seizures.

Therapeutics having anticonvulsant properties are used in the treatment of seizures. Most therapeutics used to abolish or attenuate seizures act at least through effects that reduce the spread of excitation from seizure foci and prevent detonation and disruption of function of normal aggregates of neurons. Anticonvulsants which have been utilized include phenytoin, phenobarbital, primidone, carbamazepine, ethosuximide, clonazepam and valproate. For further details of seizures and their therapy (see Rall & Schleifer (1985) and The Merck Manual (1992)).

Due to the involvement of exotoxic-dependent neurodegeneration which can result from seizure, certain hedgehog and ptc therpaeutics of the present invention may be useful as part of a regimen in the treatment of epilepsy, and are preferably used in conjunction with a treatment including an anticonvulsant agent.

VI. Exemplary Pharmaceutical Preparations

Those of skill in treating neural tissues can determine the effective amount of an therapeutic composition to be formulated in a pharmaceutical or cosmetic preparation.

These therapeutic formulations used in the method of the invention are most preferably applied in the form of appropriate compositions. As appropriate compositions there may be cited all compositions usually employed for systemically or locally (such as intrathecal) administering drugs. The pharmaceutically acceptable carrier should be substantially inert, so as not to act with the active component. Suitable inert carriers include water, alcohol polyethylene glycol, mineral oil or petroleum gel, propylene glycol and the like.

To prepare the pharmaceutical compositions of this invention, an effective amount of the particular hedgehog therapeutic and the neurotrophic factor is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules, and tablets. Because of their ease in administration, tablets and capsules represents the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations. In the compositons suitable for percutaneous administration, the carrier optionally comprises a penetration enhancing agent and/or a suitable wetting agent, optionally combined with suitable additives of any nature in minor proportions, which additives do not introduce a significant deleterious effect on the skin.

It is especially advantageous to formulate the subject compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powders packets, wafers, injectable solutions or suspensions, teaspoonfuls, tablespoonfuls and the like, and segregated multiples thereof.

The pharmaceutical preparations of the present invention can be used, as stated above, for the many applications which can be considered cosmetic uses. Cosmetic compositions known in the art, preferably hypoallergic and pH controlled are especially preferred, and include toilet waters, packs, lotions, skin milks or milky lotions. The preparations contain, besides the hedgehog therapeutic and the neurotrophic factor, components usually employed in such preparations. Examples of such components are oils, fats, waxes, surfactants, humectants, thickening agents, antioxidants, viscosity stabilizers, chelating agents, buffers, preservatives, perfumes, dyestuffs, lower alkanols, and the like. If desired, further ingredients may be incorporated in the compositions, e.g. antiinflammatory agents, antibacterials, antifungals, disinfectants, vitamins, sunscreens, antibiotics, or other anti-acne agents.

Examples of oils comprise fats and oils such as olive oil and hydrogenated oils; waxes such as beeswax and lanolin; hydrocarbons such as liquid paraffin, ceresin, and squalane; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearate, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrocloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants comprise butylated hydroxytoluene, butylated hydroxyanisole, propyl gallate, citric acid and ethoxyquin; examples of chelating agents include disodium edetate and ethanehydroxy diphosphate; examples of buffers comprise citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid.

For preparing ointments, creams, toilet waters, skin milks, and the like, typically from 0.01 to 10% in particular from 0.1 to 5% and more in particular from 0.2 to 2.5% of the active ingredient, e.g., of the hedgehog therapeutic and the neurotrophic factor, will be incorporated in the compositions. In ointments or creams, the carrier for example consists of 1 to 20%, in particular 5 to 15% of a humectant, 0.1 to 10% in particular from 0.5 to 5% of a thickener and water; or said carrier may consist of 70 to 99%, in particular 20 to 95% of a surfactant, and 0 to 20%, in particular 2.5 to 15% of a fat; or 80 to 99.9% in particular 90 to 99% of a thickener; or 5 to 15% of a surfactant, 2-15% of a humectant, 0 to 80% of an oil, very small (<2%) amounts of preservative, coloring agent and/or perfume, and water. In a toilet water, the carrier for example consists of 2 to 10% of a lower alcohol, 0.1 to 10% or in particular 0.5 to 1% of a surfactant, 1 to 20%, in particular 3 to 7% of a humectant, 0 to 5% of a buffer, water and small amounts (<2%) of preservative, dyestuff and/or perfume. In a skin milk, the carrier typically consists of 10-50% of oil, 1 to 10% of surfactant, 50-80% of water and 0 to 3% of preservative and/or perfume. In the aforementioned preparations, all % symbols refer to weight by weight percentage.

Particular compositions for use in the method of the present invention are those wherein the hedgehog or ptc therapeutic is formulated in liposome-containing compositions. Liposomes are artificial vesicles formed by amphiphatic molecules such as polar lipids, for example, phosphatidyl cholines, ethanolamines and serines, sphingomyelins, cardiolipins, plasmalogens, phosphatidic acids and cerebiosides. Liposomes are formed when suitable amphiphathic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unilamellar vesicle. If water-soluble materials are included in the aqueous phase during the swelling of the lipids they become entrapped in the aqueous layer between the lipid bilayers.

Water-soluble active ingredients such as, for example, various salt forms of a hedgehog polypeptide, are encapsulated in the aqueous spaces between the molecular layers. The lipid soluble active ingredient of hedgehog or ptc therapeutic, such as an organic mimetic, is predominantly incorporated into the lipid layers, although polar head groups may protude from the layer into the aqueous space. The encapsulation of these compounds can be achieved by a number of methods. The method most commonly used involves casting a thin film of phospholipid onto the walls of a flask by evaporation from an organic solvent. When this film is dispersed in a suitable aqueous medium, multilamellar liposomes are formed. Upon suitable sonication, the coarse liposomes form smaller similarly closed vesicles.

Water-soluble active ingredients are usually incorporated by dispersing the cast film with an aqueous solution of the compound. The unencapsulated compound is then removed by centrifugation, chromatography, dialysis or other art-known suitable procedures. The lipid-soluble active ingredient is usually incorporated by dissolving it in the organic solvent with the phospholipid prior to casting the film. If the solubility of the material in the lipid phase is not exceeded or the amount present is not in excess of that which can be bound to the lipid, liposomes prepared by the above method usually contain most of the material bound in the lipid bilayers; separation of the liposomes from unencapsulated material is not required.

A particularly convenient method for preparing liposome formulated forms of hedgehog and ptc therapeutics is the method described in EP-A-253,619, incorporated herein by reference. In this method, single bilayered liposomes containing encapsulated active ingredients are prepared by dissolving the lipid component in an organic medium, injecting the organic solution of the lipid component under pressure into an aqueous component while simultaneously mixing the organic and aqueous components with a high speed homogenizer or mixing means, whereupon the liposomes are formed spontaneously.

The single bilayered liposomes containing the encapsulated hedgehog or ptc therapeutic can be employed directly or they can be employed in a suitable pharmaceutically acceptable carrier for localized administration. The viscosity of the liposomes can be increased by the addition of one or more suitable thickening agents such as, for example xanthan gum, hydroxypropyl cellulose, hydroxypropyl methylcellulose and mixtures thereof. The aqueous component may consist of water alone or it may contain electrolytes, buffered systems and other ingredients, such as, for example, preservatives. Suitable electrolytes which can be employed include metal salts such as alkali metal and alkaline earth metal salts. The preferred metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means which is located outside the vessel. In the latter case, the liposomes are formed in the mixing means and then transferred to another vessel for collection purpose.

The organic component consists of a suitable non-toxic, pharmaceutically acceptable solvent such as, for example ethanol, glycerol, propylene glycol and polyethylene glycol, and a suitable phospholipid which is soluble in the solvent. Suitable phospholipids which can be employed include lecithin, phosphatidylcholine, phosphatydylserine, phosphatidylethanol-amine, phosphatidylinositol, lysophosphatidylcholine and phospha-tidyl glycerol, for example. Other lipophilic additives may be employed in order to selectively modify the characteristics of the liposomes. Examples of such other additives include stearylamine, phosphatidic acid, tocopherol, cholesterol and lanolin extracts.

In addition, other ingredients which can prevent oxidation of the phospholipids may be added to the organic component. Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of an hh at a particular target site. Such embodiments of the present invention can be used for the delivery of an exogenously purified hedgehog polypeptide, which has been incorporated in the polymeric device, or for the delivery of hedgehog produced by a cell encapsulated in the polymeric device.

An essential feature of certain embodiments of the implant can be the linear release of the therapeutic, which can be achieved through the manipulation of the polymer composition and form. By choice of monomer composition or polymerization technique, the amount of water, porosity and consequent permeability characteristics can be controlled. The selection of the shape, size, polymer, and method for implantation can be determined on an individual basis according to the disorder to be treated and the individual patient response. The generation of such implants is generally known in the art. See, for example, Concise Encylopedia of Medical & Dental Materials, ed. by David Williams (MIT Press: Cambridge, Mass., 1990); and the Sabel et al. U.S. Pat. No. 4,883,666.

In another embodiment of an implant, a source of cells producing the therapeutic, e.g., secreting a soluble form of a hedgehog polypeptide, is encapsulated in implantable hollow fibers or the like. Such fibers can be pre-spun and subsequently loaded with the cell source (Aebischer et al. U.S. Pat. No. 4,892,538; Aebischer et al. U.S. Pat. No. 5,106,627; Hoffman et al. (1990) Expt. Neurobiol. 110:39-44; Jaeger et al. (1990) Prog. Brain Res. 82:41-46; and Aebischer et al. (1991) J. Biomech. Eng. 113:178-183), or can be co-extruded with a polymer which acts to form a polymeric coat about the cells (Lim U.S. Pat. No. 4,391,909; Sefton U.S. Pat. No. 4,353,888; Sugamori et al. (1989) Trans. Am. Artif Intern. Organs 35:791-799; Sefton et al. (1987) Biotehnol. Bioeng. 29:1135-1143; and Aebischer et al. (1991) Biomaterials 12:50-55).

VII. Examples:

Cholinergic Neuron Cultures

The culture system to study the effects of sonic hedgehog on cholinergic neurons is a mixed culture system containing both neurons and glia at different stages of maturation. This lends itself to three possible explanations of observed effects on these neurons. The first possibility is that Shh has an effect on neuronal precursors that are present in the cultures. Proliferation, differentiation, or survival can be measured on precursor populations. Shh influences precursor proliferation and differentiation in the spinal cord (Dutton et al., 1999) and proliferation in cerebellar granular cells (Wechsler-Reya and Scott, 1999). Sonic hedgehog has also been shown to promote phenotypic specification in vitro of precursors by the induction of spinal motor neurons (Roelink et al., 1994; Tanabe et al., 1995), midbrain dopaminergic neurons (Hynes et al., 1995; Wang et al., 1995), and basal forebrain cholinergic neurons (Ericson et al., 1995).

The second effect on these cultures could be on the mature cholinergic neurons. Neurotransmitter regulation measured by CHAT expression is one example of how to categorize what is happening in the mature cell. Enhanced survival is also an indication of an effect on mature cells. Sonic hedgehog has been shown to have effects on mature. neurons in culture by eliciting a protective effect in response to toxic injury (Miao, et al., 1997).

Lastly, sonic hedgehog may have an indirect effect mediated through the glia in these cultures. The glia may secrete a factor in response to hedgehog that may have effects on the cholinergic neurons. These three possibilities would be interesting and novel for this population of neurons.

Cell Dissociation and Culture

The medial septum region was removed from embryonic day 16 rat embryos. All the septi were collected in Dulbeccos's Modified Eagle's Medium (DMEM) and kept on ice during the length of time to complete the dissection. Tissue was dissociated in 0.1% Trypsin-EDTA for 20 minutes in a 37° C. waterbath. The dissociation solution was removed and replaced with 0.05% Dnase/10% fetal bovine serum in DMEM with L-glutarnine and penicillin-streptomycin. The dissociated tissue was washed a total of three times for ten minutes each. Flame polished Pasteur pipettes of graded bore sized were used to gently triturate the tissue into a single cell suspension. Cells were plated in 4well LabTek chamber slides at a density of 1.5×10⁵ cells/cm² in growth medium. Standard growth medium was DMEM with 10% fetal bovine serum, L-glutamine, and penicillin-steptomycin. Cultures grown in serum free conditions were grown in a different medium. This medium consisted of DMEM/F-12 mixed with Neurobasal medium at a 2:1 ration supplemented with B-27 and penicillin-streptomycin. Slides were previously coated with a 0.05% solution of poly-1-lysine to enhance cell attachment. Cultures were fed every two days by removal of half of the medium and replenishment of growth factors. The culture period took place in a 37° C. humidified chamber supplied with 5% carbon dioxide and ranged from 7-14 days.

Immunohistochemical Analysis

After the predetermined culture period, cells were fixed with 4% parafonnaldehyde in phosphate buffered saline(PBS) for twenty minutes at room temperature. Cultures were then washed three times with PBS and incubated in a blocking solution to decrease staining background. The blocking solution consisted of 3% bovine serum albumin, 0.1% Triton-X100 detergent, and 3% serum specific to the species that the secondary antibody was raised in. After blocking for 30 minutes at room temperature, cultures were incubated with primary antibodies against either rabbit anti-goat ChAT, mouse anti-mouse NeuN, or mouse anti-rat BrdU overnight at 4° C. After washing three times with PBS, the cells were incubated with the secondary antibody for one hour at room temperature. These antibodies are conjugated to either cyanin3 or flourescein for. visualization with the appropriate optics. The staining to recognize BrdU included a treatment with 2N hydrochloric acid followed by 0.1 M borate buffer to permeabilize the cells. All dilutions of the primary and secondary antibodies were made in the blocking solution. After the final set of washes the slides were coverslipped in a mounting medium containing an agent to protect the staining from fading and to label all the nuclei of the cells.

Measuring Proliferation

Proliferation in the cultures was measured by two methods. The cultures were labeled with either bromodeoxyuridine(BrdU) or tritiated thymidine(³H-thymidine) to detect the cells that were in the process of division at the time the agents were added. The nuclei of dividing cells will incorporate either agent to allow the cell to be visualized by the appropriate technique. Detecting cell proliferation by the use of BrdU was first pursued. The cultures were plated as usual and 1 OmM BrdU was added at two days in vitro. The cultures were fixed and stained on day three. Cells positive for BrdU were scored. This was done for cultures grown with and without serum. The other agent used to detect cells that were proliferating was ³H-thymidine. This was done by the addition Of 1uCi of ³H-thymidine on day two or day six of the culture period. The cultures were returned to the incubator for six hours after which time the ³H-thymidine was washed out. After eight days in vitro the cultures were fixed and stained for analysis.

Visualization of ³H-thymidine /ChAT Cells

After eight days in vitro, the cultures were fixed with 4% paraformaldehyde and stained. for ChAT. Following the primary antibody incubation, the cultures were incubated using a kit containing an avidin-biotin complex following the directions of the manufacturer (Vector). A diaminobenzidine substrate (Dako) was used to visualize the cells by the presence of a brown precipitate that forms over the ChAT positive cells. The cultures were post fixed with 4% paraformaldehyde and then dipped in a 2% gelatin solution to decrease and prevent any background in the autoradiography to follow. Slides were allowed to dry horizontally in a dust-free box overnight. Slides were then dipped in autoradiography emulsion, NTB-2, that was melted and maintained at 37° C. After a steady controlled dip, the slides were again left to dry overnight in a dust-free, light safe box until they were transferred to 4 degrees. A one week incubation was sufficient to produce a strong signal of many heavily labeled cells that were apparent after development with Dektol developer and fixation in Rapid fix (Kodak).

Quantitating the Number of Positive Cells

Once the chamber slides were stained for the markers of interest, they were scored by counting the number of positive cells in the entire well. This was accomplished by encoding the wells and scoring the them blinded to the experimental conditions. Slides that were analyzed for BrdU were counted in a slightly different manor. Nine fields were randomly chosen from each well and the number of BrdU cells were scored as compared to the total number of live cells. This yielded a percent of proliferation in the cultures and controlled for any plating inconsistencies in the wells. A. Nikon compound microscope was used to examine the cells that was equipped with the appropriate optics. Once all the slides were scored, data was tabulated and analyzed using Minitab and graphed using Excel. A Students t-test was performed to assess whether any differences seen between groups was statistically significant.

Cooperation of Shh and NGF

The effects of Shh on basal forebrain cholinergic neurons was examined using cultures derived from E16 rat. NGF has previously been shown to act as a survival factor, upregulating ChAT and promoting neurite outgrowth in these cultures (Hartikka, 1988). This result demonstrated a two-fold increase in ChAT positive cells. When Shh is added alone to these cultures, there was no detectable change as measured by cell scoring of the immunochernically labeled cells. The combined addition of Shh and NGF to the cultures resulted in a three to four-fold increase in the number of ChAT positive cells. These cultures were replenished every two days with fresh factors and initially grown for fourteen days in vitro. Cells in these conditions have an enlarged appearance over the control cells with many branching processes.

Temporal Effects of Shh and NGF

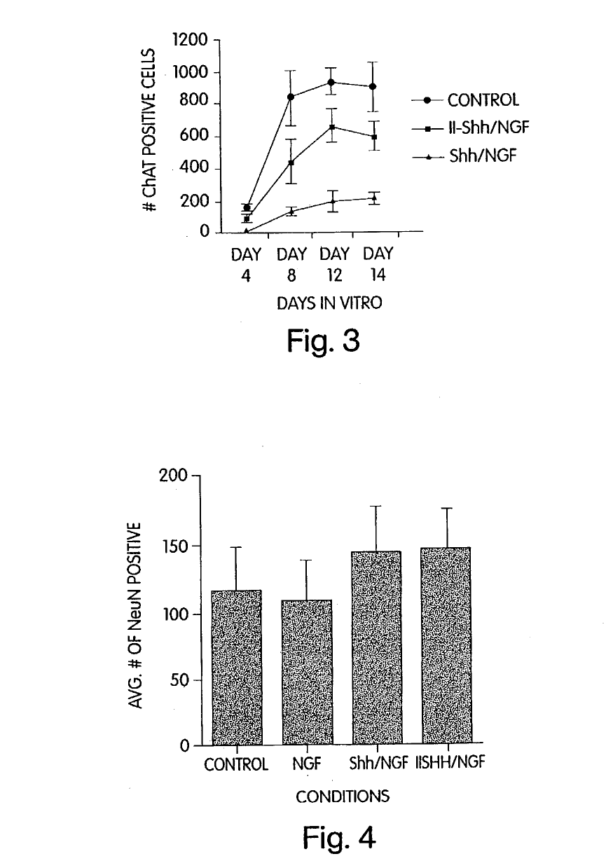

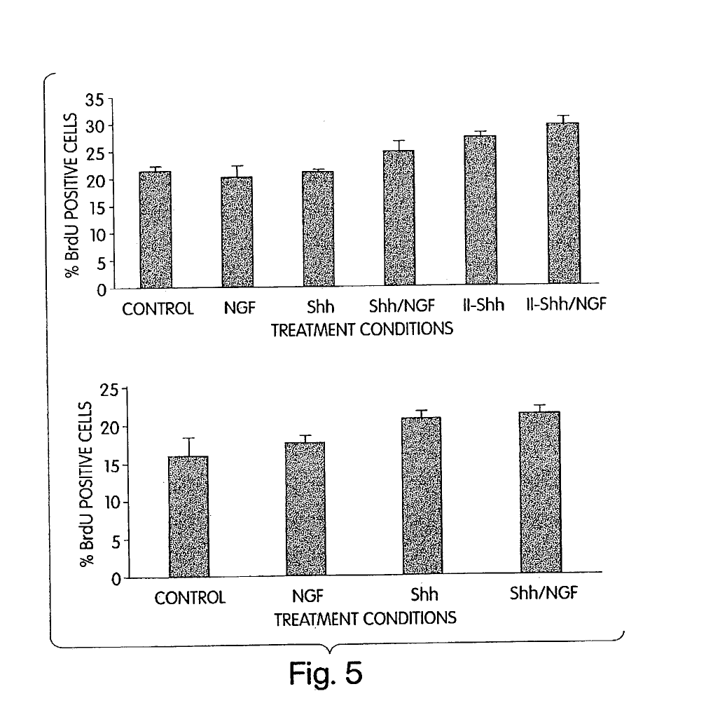

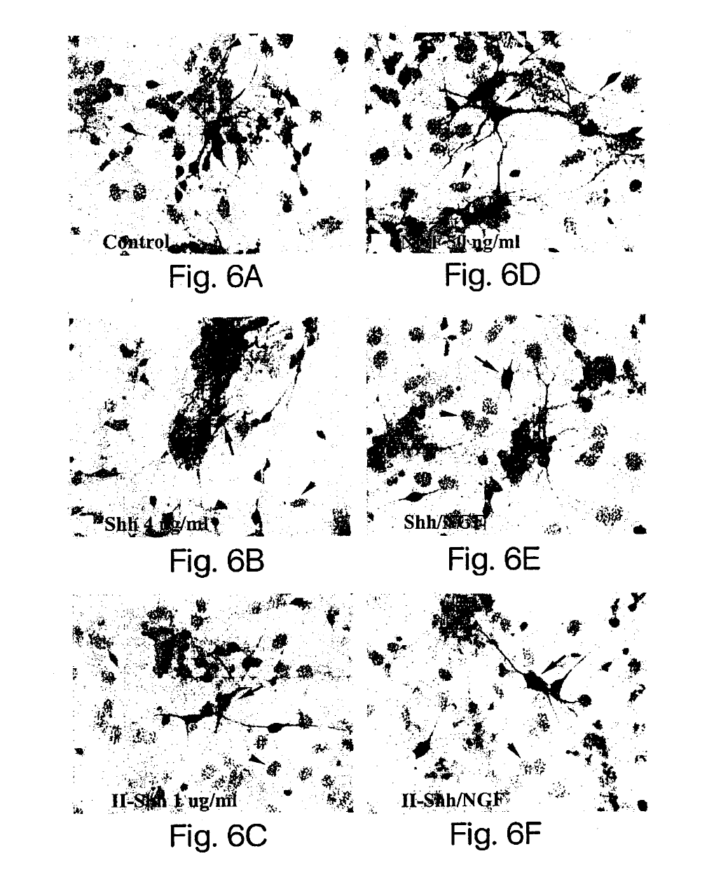

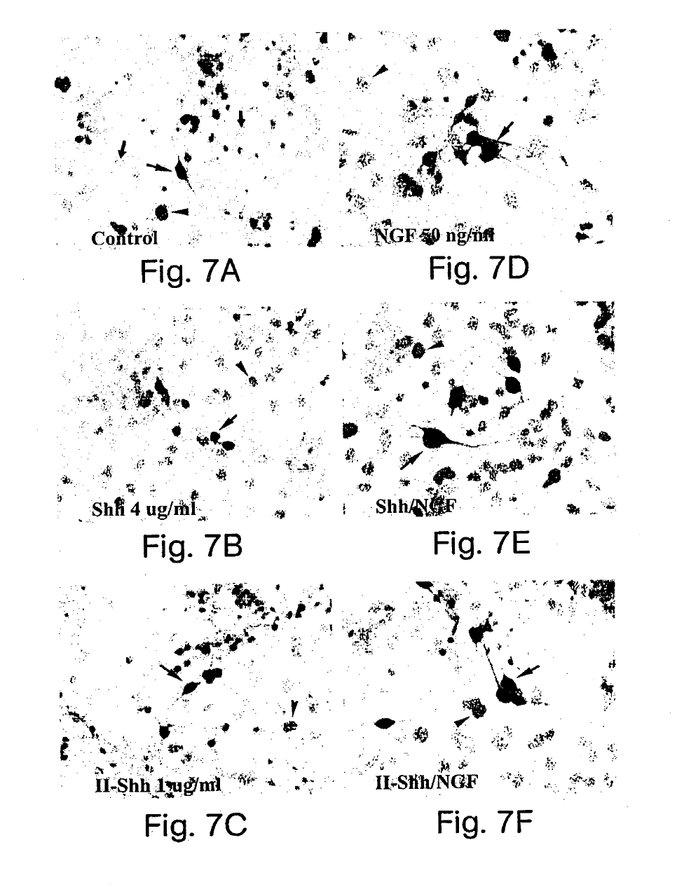

It was initially interesting to determine what was the actually temporal development of the cultures. This would determine if the cultures could be grown for a shorter period of time with the same result. Cultures were plated and subsets were fixed at intervals of the culture period which included 4, 8, 12, and 14 days in vitro. By day 8 of the culture development a four-fold increase can be observed when both Shh and NGF are present (FIG. 3). It seems to plateau and begin to decline by day 14 possibly demonstrating cell death. This indicated that it would be possible to shorten the length of the culture period.

Precursor Proliferation

One possible explanation for the observed affect could be the result of precursor proliferation. This expanded precursor pool would lead to more neurons and therefore more cholinergic neurons. Cultures were grown for eight days in vitro then were fixed and stained for the neural marker NeuN and scored for the number of positive cells. This nuclear marker labels all types of neurons. There is a statistically significant increase in the number of NeuN positive cells when the cultures are grown in the presence of NGF. and Shh as compared with NGF alone or control(FIG. 4). This suggests either an increase in the number of precursors that are proliferating in response to the growth factors or a survival effect on the neurons that are normally present.

BrdU Incorporation

Cultures were established as before and were tested for the incorporation of BrdU as an indicator of cell division. Cultures grown in ten percent serum were labeled overnight on day two with BrdU. The cells were then stained and scored for the number of BrdU positive cells present in the experimental conditions. Significantly more positive cells were counted in the conditions that contained Shh along with NGF. The initial incorporation of BrdU was tested on cultures that were grown in the standard growth medium that contained ten percent fetal bovine serum. Since these cultures are a mixed population, containing neurons as well as glia, the observed increase in proliferation could be attributed to the dividing glia. Since the result was not as significant a fold increase that was observed with the addition of Shh and NGF measuring the ChAT positive neurons, it was necessary to remove the serum from the cultures. Serum free cultures were established with defined medium and BrdU was added in the same paradigm. Again a significant increase was noted with the addition of Shh and NGF although it was still quite subtle.

Thymidine Incorporation

³H-thymidine was added to cholinergic neuron cultures that had been established by the methods outlined earlier. Cultures were pulsed and then rinsed and left to grow. for eight days today. Two subsets received the pulse either on day two or day six. This would potentially label any cells that are proliferating early in the culture period as well as late in the culture period. Staining the cultures for ChAT allowed for the cholinergic neurons to be visualized. The autoradiography indicates cells that were proliferating at the time of pulse by the presence of silver grains over the dividing cells. All of the conditions have many cells that are heavily labeled with silver grains indicating that there is proliferation occurring. However, of the heavily labeled cells none of them are also positive for CHAT. It was difficult to find any cholinergic neurons that were also positive for silver grains.

Auburger, et al. (1987) Developmental changes of nerve growth factor and its “mRNA in the rat hippocampus: comparison with choline acetyltransferase. Developmental Biology, 120: 322-328.

Chiang, C. et al. (1996) Cyclopia and defective patterning in mice lacking sonic hedgehog gene function. Nature, 383: 407-413.

Dutton, R., et al. (1999) Sonic hedgehog promotes neuronal differentiation of murine spinal cord precursors and collaborates with neurotrophin 3 to induce islet-1. Journal of Neuroscience 19: 2601-2608.

Ericson, J., et al. (1995) Sonic hedgehog induces the differentiation of ventral forebrain neurons: a common signal for ventral patterning within the neural tube. Cell, 18: 747-756.

Gilmor, M. L. et al., (1999) Preservation of nucleus basalis neurons containing choline acetyltransferase and the vesicular acetylcholine transporter in the elderly with mild cognitive impairment and early Alzheimer's disease. Journal of Comparative Neurology, 411: 693-704.

Hartikka, J., and Hefti, F. (1988) Development of septal cholinergic neurons in culture:

plating density and glial cell modulate effects of NGF on survival, fiber growth,. and expression of transmitter-specific enzymes. Journal of Neuroscience, 8: 2967-2985.

Hashimoto, Y. et al. (1999) Synergistic effects of brain-derived neurotrophic factor and ciliary neurotrophic factor on cultured basal forebrain cholinergic neurons from postnatal 2-week-old rats. Developmental Brain Research, 115: 25-32.

Hefti, F. (1986) Nerve growth factor promotes survival of septal cholinergic neurons after fimbrial transections. Journal of Neuroscience, 6: 2155-62.

Hynes, M., et al. (1995) Induction of midbrain doparninergic neurons by sonic hedgehog. Neuron, 15: 35-44.

Lehericy, S. et al. (1993) Heterogeneity and selectivity of the degeneration of cholinergic neurons in the basal forebrain of patients with Alzheimer's disease. Journal of Comparative Neurology, 330: 15-31.

Johnson, R. L. and Scott, M. P. (1998) New players and puzzles in the hedgehog signaling pathway. Current Opinion in Genetics & Development, 8: 450-456.

Miao, N., et al. (1997) Sonic hedgehog promotes the survival of specific CNS populations and protects these cells from toxic insult in vitro. Journal of Neurosciene, 17: 5891-5899.

Mufson, E. J., et al. (1998) Galanin expression within the basal forebrain in Alzheimer's disease. Comments on therapeutic potential. Annals of NY Academy of Science, 863: 291-304.

Peterson, G. M. (1990) Fate of septohippocampal. neurons following fimbria-fornix transection: a time course analysis. Brain Research Bulletin, 25: 128-37.

Roelink, H. et al. (1994) Floor plate and motor neuron induction by Vhh-1, a vertebrate homologue of hedgehog expressed by the notochord. Cell, 76: 761-775.

Sramek J. J. and Cutler, N. R. (1999) Recent developments in the drug treatment of Alzheimer's disease, Drugs and Aging, 14: 359-73.

Svendsen, C. (1995) Septal neurons. In J. Cohen & G. P. Wilkins(Eds.) Neural cell culture a practical approach (pp4l-53).Oxford: IRL Press.

Tanabe, Y., et al. (1995) Induction of motor neurons by sonic hedgehog is independent of floor plate differentiation. Current Biology, 5: 651-658.

Wang, M. Z., et al. (1995) Induction of the doparninergic neuron phenotype in the midbrain by sonic hedgehog protein. Nature Medicine, 1: 1184-1188.

Weshsler-Reya, R. J. and Scott M. P. (1999) Control of neuronal precursor proliferation in the cerebellum by Sonic Hedgehog. Neuron, 22: 103-14.

Brady, D. R., Phelps, P. E., and Vaughn, J. E. (1989). Neurogenesis of basal forebrain cholinergic neurons in rat. Dev Brain Res 47(1): 81-92.

Chiang, C., Litingtung, Y., Lee, E., Young, K. E., Corden, J. L., Westphal, H., and Beachy, P. A. (1996). Cyclopia and defective patterning in mice lacking sonic hedgehog gene function. Nature 383: 407-413.

Dale, J. K., Vesque, C., Lints, T. J., Sampath, T. K., Furley, A., Dodd, J., and Placzek, M. (1997). Cooperation of BMP7 and SHH in the induction of forebrain ventral midline cells by prechordal mesoderm. Cell 90(2): 257-69.

Dutton, R., Yamada, T., Turnley, A., Bartlett, P. F., and Murphy, M. (1999). Sonic Hedgehog Promotes Neuronal Differentiation of Murine Spinal Cord Precursors and Collaborates with Neurotrophin 3 to Induce Islet-1. J Neurosci 19: 2601 -2608.

Ericson, J., Muhr, J., Placzek, M., Lints, T., Jessell, T. M., and Edlund, T. (1995). Sonic Hedgehog Induces the Differentiation of Ventral Forebrain Neurons: A Common Signal for Ventral Patterning within the neural tube. Cell 18: 747-756.

Gilmor, M. L., Erickson, J. D., Varoqui, H., Hersh, L. B., Bennett, D. A., Cochran, E. J., Mufson, E. F., and Levey, A. I. (1999). Preservation of nucleus basalis neurons containing choline acetyltransferase and the vesicular acetylcholine transporter in the elderly with mild cognitive impairment and early Alzheimer's disease. J Comp Neurol 411: 693-704.

Goodrich, L. V., Johnson, R. L., Milenkovic, L., McMahon, J. A., and Scott, M. P. (1996). Conservation of the hedgehog/patched signaling pathway from flies to mice: induction of a mouse patched gene by Hedgehog. Genes Dev. 10(3): 301-12.

Goodrich, L. V., Milenkovic, L., Higgins, K. M., and Scott, M. P. (1997). Altered Neural Cell Fates and Medulloblastoma in Mouse patched Mutants. Science 277: 1109-1113.

Gunhaga, L., Jessell, T. M., and Edlund, T. (2000) Sonic hedgehog signaling at gastrula stages specifies ventral telencephalic cells in the chick embryo. Development 127(15): 3283-93

Hartikka, J., and Hefti, F. (1988). Development of septal cholinergic neurons in culture: plating density and glial cell modulate effects of NGF on survival, fiber growth, and expression of transmitter-specific enzymes. J Neurosci 8: 2967-2985.

Hellweg, R., Gericke, C. A., Jendroska, K., Hartung, H. D., and Cervos-Navarro, J. (1998). NGF content in the cerebral cortex of non-demented patients with amyloid-plaques and in symptomatic Alzheimer's disease. Int J Dev Neurosci 16(7-8): 787-794.

Hock, C., Heese, K., Hulette, C., Rosenberg, C., and Otten, U. (2000). Region-specific neurotrophin imbalances in Alzheimer disease: decreased levels of brain-derived neurotrophic factor and increased levels of nerve growth factor in hippocampus and cortical areas. Arch Neurol: 57(6): 846-851.

Hynes, M., Porter, J. A., Chiang, C., Dhang, D., Tessier-Lavigne, M., Beachy, P. A., and Rosenthal, A. (1995a). Induction of midbrain dopaminergic neurons by sonic hedgehog. Neuron 15: 35-44.

Hynes, M., Poulsen, K., Tessier-Lavigne, M., and Rosenthal, A. (1995b) Control of neuronal diversity by the floor plate: contact-mediated induction of midbrain dopaminergic neurons. Cell. 80(1): 95-101.

Jensen, A. M., and Wallace, V. A. (1997). Expression of Sonic hedgehog and its putative role as a precursor cell mitogen in the developing mouse retina. Development. 124(2): 363-371.

Kalyani, A. J., Piper, D., Mujtaba, T., Lucero, M. T., and Rao, M. S. (1998). Spinal cord neuronal precursors generate multiple neuronal phenotypes in culture. J Neurosci 18(19): 7856-7868.

Lehericy, S., Hirsch, E. C., Cervera-Pierot, P., Hersh, L. B., Bakchine, S., Piette, F., Duyckaerts, C., Hauw, J. J., Javoy-Agid, F., Agid, Y. (1993). Heterogeneity and selectivity of the degeneration of cholinergic neurons in the basal forebrain of patients with Alzheimer's disease. J Comp Neurol 330: 15-31.

McMahon, A. P. (2000). More Surprises in the Hedgehog Signaling Pathway. Cell 100: 185-188.

Miao, N., Wang, M., Ott, J. A., D'Alessandro, J. S., Woolf, T. M., Bumcrot, D. A., Mahanthappa, N. K., and Pang, K. (1997). Sonic Hedgehog Promotes the Survival of Specific CNS Populations and Protects These Cells from Toxic Insult In Vitro. J Neurosci 17: 5891-5899.

Pepinsky, R. B., Rayhorn, P., Day, E. S., Dergay, A., Williams, K. P., Galdes, A., Taylor, F. R., Boriack-Sjodin, P. A., Garber, E. A. (2000). Mapping Sonic Hedgehog-Receptor Interactions by Steric Interference. J Biol Chem 275: 10995-11001.

Roelink, H., Augsburger, A., Heemskerk, J., Korzh, V., Norlin, S., Ruiz i Altaba, A., Tanabe, Y., Placzek, M., Edlund, T., Jessell, T. M. et al (1994). Floor plate and motor neuron induction by Vhh-1, a vertebrate homologue of hedgehog expressed by the notochord. Cell 76: 761-775.

Roelink, H., Porter, J. A., Chiang, C., Tanabe, Y., Chang, D. T., Beachy, P. A., Jessell, T. M. (1995). Floor plate and motor neuron induction by different concentrations of the amino-terminal cleavage product of sonic hedgehog autoproteolysis. Cell 81(3): 445-455.

Roessler, E., Belloni, E., Gaudenz, K., Jay, P., Berta, P., Scherer, S. W., Tsui, L. C., Muenke, M. (1996). Mutations in the human Sonic Hedgehog gene cause holoprosencephaly. Nat Genet. 14(3): 357-360.

Rowitch, D. H., St.-Jacques, B., Lee, S. M. K., Flax, J. D., Snyder, E. Y., and McMahon, A. P. (1999). Sonic hedgehog Regulates Proliferation and Inhibits Differentiation of CNS Precursor Cells. J Neurosci 19(20): 8954-8965.

Schotzinger, R. J., and Landis, S. C. (1990). Acquisition of cholinergic and peptidergic properties by sympathetic innervation of rat sweat glands requires interaction with normal target. Neuron 5(1): 91-100.

Svendsen, C. N. (1995). Septal neurons. In: Neural Cell Culture a practical approach (Cohen J, Wilkins G P, eds), pp 41-53. Oxford: IRL Press.

Svendsen, C. N., Kew, J. N., Staley, K., Sofroniew, M. V. (1994). Death of developing septal cholinergic neurons following NGF withdrawal in vitro: protection by protein synthesis inhibition. J Neurosci 14: 75-87.

Tanabe, Y., Roelink, H., and Jessell, T. M. (1995). Induction of Motor Neurons by Sonic Hedgehog is Independent of Floor Plate Differentiation. Curr Biol 5: 651-658.

Taylor, F. R., Wen, D., Garber, E. A., Carmillo, A. N., Baker, D. P., Arduini, R. M., Williams, K. P., Weinreb, P. H., Rayhorn, P., Hronowski, X., Whitty, A., Day, E. S., Boriack-Sjodin, A., Shapiro, R. I., Galdes, A., and Pepinsky, R. B. (2001). Enhanced potency of human Sonic hedgehog by hydrophobic modification. Biochemistry 40(14): 4359-4371.

Traiffort, E., Charytoniuk, D., Watroba, L., Faure, H., Sales, N., and Ruat, M. (1999). Discrete localizations of hedgehog signalling components in the developing and adult rat nervous system. Eur J Neurosci 11: 3199-3214.

Wang, M. Z., Jin, P., Bumcrot, D. A., Marigo, V., McMahon, A. P., Wang, E. A., Woolf, T., and Pang, K. (1995). Induction of the dopaminergic neuron phenotype in the midbrain by sonic hedgehog protein. Nature Med 1: 1184-1188.

Wechsler-Reya, R. J. and Scott, M. P. (1999). Control of neuronal precursor proliferation In the cerebellum by Sonic Hedgehog. Neuron 22: 103-14.

Wilkinson, D. G., Bailes, J. A., and McMahon, A. P. (1987). Expression of the proto-oncogene int-1 is restricted to specific neural cells in the developing mouse embryo. Cell 50: 79-88.

Williams, K. P., Rayhorn, P., Chi-Rosso, G., Garber, E. A., Strauch, K. L., Horan, G. S., Reilly, J. O., Baker, D. P., Taylor, F. R., Koteliansky, V., Pepinsky, R. B. (1999). Functional antagonists of sonic hedgehog reveal the importance of the N terminus for activity. J Cell Sci. 112: 4405-4414.

Ye, W., Shimamura, K., Rubenstein, J. L., Hynes, M. A., Rosenthal, A. (1998). FGF and Shh signals control dopaminergic and serotonergic cell fate in the anterior neural plate. Cell 93(5): 755-66. Works Consulted

Echelard, Y., et al. (1993) Sonic hedgehog, a member of a family of putative signaling molecules, is implicated in the regulation of CNS polarity. Cell, 75: 1417-1430.

Hammerschmidt, M., et al. (1997) The world according to hedgehog. Trends in Genetics, 13: 14-21.

Johnson, R. L. and Tabin, C. (1995) The long and short of hedgehog signaling. Cell, 81: 313-316.

Lewin, G. R. and Barde, Y -A. (1996) Physiology of the neurotrophins. Annual Review of Neuroscience, 19: 289-317.

Marti, E., etal. (1995) Requirement of 19 k form of sonic hedgehog for induction of distinct ventral cell type in CNS explants. Nature, 375: 322-325.

Svendsen, C. N., et al. (1994) Death of developing septal cholinergic neurons following NGF withdrawal in vitro: protection by protein synthesis inhibition. Journal of Neuroscience, 14: 75-87.

Traiffort, E., ct al. (1998) Regional distribution of sonic hedgehog, patched, and smoothened mRNA in the adult rat brain. Journal of Neurochemistry, 70:1327-1330.

Wallace, V. A. and Raff, M. C. (1999) A role for sonic hedgehog in axon-to-astrocytc signaling in the rodent optic nerve. Development,126: 2901-2909.

What is claimed:

1. A method of ameliorating neuronal degeneration in a subject comprising administering to the subject a trophic amount of a composition comprising a hedgehog therapeutic and a neurotrophic factor.

2. The method of claim 1, wherein the hedgehog therapeutic comprises a full length mature hedgehog polypeptide or a bioactive fragment of a hedgehog polypeptide.

3. The method of claim 2, wherein the sequence of the hedgehog therapeutic comprises at least the sequence of residue 24-197 of SEQ ID No. 15 or resideus 28-202 of SEQ ID No. 16 or residues 28-202 of 23-198 of SEQ ID No. 17.

4. The method of claim 2, wherein the hedgehog therapeutic comprises a naturally occurring hedgehog polypeptide or a naturally occurring bioactive fragment thereof.

5. The method of claim 1, wherein the hedgehog therapeutic is administered to the subject by administering a viral expression vector for expressing a hedgehog therapeutic.

6. The method of claim 1, wherein there is neuronal degeneration of the cholinergic neurons of the basal forebrain of the subject.

7. The method of claim 1, wherein the subject suffers from or suffers from a predisposition for Alzheimer's Disease.

8. The method of claim 1, wherein there is neuronal degeneration of the dopaminergic or GABAergic neurons.

9. The method of claim 1, wherein the subject suffers from or suffers from a predisposition for Parkinson's Disease, Huntingdon's Chorea or Amyotrophic Lateral Sclerosis.

10. The method of claim 1, wherein the composition comprises a factor selected from the group consisting of IGF-1, active truncated derivatives thereof, aFGF, bFGF, PDGF, GDNF, NT-3 and NT-4/5.

11. The method of claim 1, wherein the composition comprises NGF.

12. The method of claim 11, wherein the neurotrophic factor consists essentially of NGF.

13. The method of claim 1, wherein the composition comprises BDNF or CNTF.

14. The method of claim 13, wherein the neurotrophic factor consists essentially of BDNF, CNTF or a mixture thereof.

15. A kit comprising a hedgehog therapeutic in a pharmaceutically acceptable carrier and a neurotrophic factor in a pharmaceutically acceptable carrier.

16. The kit of claim 15, which further comprises a label that indicates the kit can be used in the treatment, amelioration or prevention of neuronal degeneration in a subject.

17. The kit of claim 16, in which the label indicates the kit can be used in the treatment, amelioration or prevention of Alzheimer's Disease.

18. The kit of claim 16, in which the label indicates the kit can be used in the treatment, amelioration or prevention of either Parkinson's Disease, Huntingdon's Chorea or Amyotrophic Lateral Sclerosis.

19. The kit of claim 16, in which the hedgehog therapeutic and the neurotrophic factor form a mixture.

20. The kit of claim 16, in which the wherein the hedgehog therapeutic comprises a full length mature hedgehog polypeptide or a bioactive fragment of a hedgehog polypeptide.

21. The kit of claim 20, in which the sequence of the hedgehog therapeutic comprises at least the sequence of residues 24-197 of SEQ ID No. 15 or residues 28-202 of SEQ ID No. 16 or residues 28-202 of 23-198 of SEQ ID No. 17.

22. The kit of claim 16, in which the hedgehog therapeutic comprises a naturally occurring hedgehog polypeptide or a naturally occurring bioactive fragment thereof.

23. The kit of claim 16, in which the hedgehog therapeutic is a viral expression vector for expressing a hedgehog therapeutic.

24. The kit of claim 16, in which the neorotrophic factor comprises NGF.

25. The kit of claim 24, in which the neurotrophic factor consists essentially of NGF.

26. The kit of claim 15, in which the neurotrophic factor comprises BDNF or CNTF.

27. The kit of claim 15, in which the neurotrophic factor consists essentially of BDNF, CNTF or a mixture thereof.

28. The kit of claim 15, in which the neurotrophic factor comprises a factor selected from the group consisting of IGF-1, active truncated derivatives thereof, aFGF, bFGF, PDGF, GDNF, NT-3 and NT-4/5.
